(19) United States
(12) Patent Application Publication
(10) Pub. No.: US 2020/0163988 A1
(43) Pub. Date: May 28, 2020

(54) Immunosuppression-Reverting Oligonucleotides Inhibiting the Expression of IDO

(71) Applicant: Secarna Pharmaceuticals GmbH & Co. KG, Marburg (DE)

(72) Inventors: Richard KLAR, Munich (DE); Frank JASCHINSKI, Puchheim (DE)

(21) Appl. No.: 16/340,292

(22) PCT Filed: Oct. 9, 2017
(86) PCT No.: PCT/EP2017/075674

§ 371 Date: Apr. 8, 2019

(30) Foreign Application Priority Data
Oct. 7, 2016 (EP) 16192803.1
Aug. 24, 2017 (EP) 17187775.6

(51) Int. Cl. A61K 31/7125; C12N 15/113; A61P 37/02

(52) U.S. Cl. CPC A61K 31/7125; A61P 37/02; C12N 15/1137

(57) ABSTRACT

The present invention refers to immunosuppression-reverting oligonucleotides comprising 12 to 18 nucleotides, wherein at least one of the nucleotides is modified, and the oligonucleotide hybridizes with a nucleic acid sequence of indoleamine-2,3-dioxygenase (IDO-1) of SEQ ID NO.1 (human) in a hybridizing active area, wherein the oligonucleotide inhibits at least 50% of the IDO-1 expression. The invention is further directed to a pharmaceutical composition comprising such oligonucleotide.

The present disclosure refers to an immunosuppression-reverting oligonucleotide hybridizing with a nucleic acid sequence of indoleamine-2,3-dioxygenase (IDO) such as IDO1 and to a pharmaceutical composition comprising such immunosuppression-reverting oligonucleotide and a pharmaceutically acceptable carrier, excipient and/or dilutant.

### TECHNICAL BACKGROUND

In recent years the treatment of several different diseases such as malignant tumors was very successful by application of immune therapy, in particular by inhibitors of so called “immune checkpoints”. These checkpoints are molecules in the immune system that either turn up (co-stimulatory molecules) or down a signal. The concept of the therapeutic approach is based on the activation of endogenous anti-tumor immune reactions. Many cancers for example protect themselves from the immune system by inhibiting T cell and NK cell activity, respectively. Immune checkpoint modulators, i.e., stimulators or inhibitors are for example directed to one or more of CTLA-4, PD-1, PD-L1, LAG-3, VISTA, A2AR, BTLA, IDO, CD39, CD73, STAT3, TDO2, TIM-3, MICA, NKG2A, KIR, TIGIT, TGF-beta, Ox40, GITR, CD27, CD160, 2B4 and 4-1BB.

Tryptophan for example is an amino acid which is essential for cell proliferation and survival. It is required for the biosynthesis of the neurotransmitter serotonin, the synthesis of the cofactor nicotinamide adenine dinucleotide (NAD), and is an important component in the immune system response (“immune escape”) to tumors. Depletion of levels of tryptophan is associated with adverse effects on the proliferation and function of lymphocytes and diminished immune system response.

The enzyme indoleamine-2,3-deoxygenase (IDO) is an intracellular enzyme and it is overexpressed in many human tumors or in suppressive immune cells. IDO catalyzes the initial, rate-limiting step in the conversion of tryptophan to kynurenine resulting in lack of tryptophan and severe immunosuppressive effects of kynurenines. These effects result in suppression for example of T-cells and natural killer (NK) cells against tumor cells for example with regard to cell proliferation, cytokine secretion and/or cytotoxic reactivity. In addition, IDO expression results for example in dendritic cells in the induction of regulatory T-cells which represent a negative prognostic factor in tumor diseases. Thus, IDO is a highly relevant immunosuppressive factor for example in the tumor environment. Moreover, IDO has been implicated in neurologic and psychiatric disorders including mood disorders as well as other chronic diseases characterized by IDO activation and tryptophan degradation such as viral infections, for example, AIDS, Alzheimer's disease, cancers including T-cell leukemia and colon cancer, autoimmune diseases, diseases of the eye such as cataracts, bacterial infections such as Lyme disease, and streptococcal infections.

Small molecules such as 1-methyl-D-tryptophan have been developed and tested in clinical trials. However, 1-methyl-D-tryptophan for example shows an increase in the expression of IDO mRNA and protein due to a feedback mechanism by enzymatic inhibition of IDO. Thus, the activity of the small molecules and their in vivo half-life is limited.

Immune therapies have resulted in long-term remission, but only of small patient groups so far. The reason may be that numerous immune checkpoints and optionally further immunosuppressive mechanisms are involved in the interaction between for example the immune system and the tumor cells. The combination of immune checkpoints and potential other mechanisms may vary depending on the tumor and individual conditions of a subject to escape the body's defenses.

For the inhibition of several immunosuppressive mechanisms common approaches using an antibody and/or a small molecule are not or hardly suitable as the molecular target is located intracellularly or does not have enzymatic activity. Accordingly, an agent which is safe and effective in inhibiting the function of an “immune checkpoint” such as IDO would be an important addition for the treatment of patients suffering from diseases or conditions affected for example by the activity of this enzyme.

Oligonucleotides of the present invention are very successful in the inhibition of the expression and activity of IDO, respectively. The mode of action of an oligonucleotide differs from the mode of action of an antibody or small molecule, and oligonucleotides are highly advantageous regarding for example

(i) the penetration of tumor tissue in solid tumors,
(ii) the blocking of multiple functions and activities, respectively, of a target,
(iii) the combination of oligonucleotides with each other or an antibody or a small molecule, and
(iv) the inhibition of intracellular effects which are not accessible for an antibody or inhibitable via a small molecule.

### SUMMARY

The present invention refers to an oligonucleotide such as an immunosuppression-reverting oligonucleotide comprising about 10 to 20 nucleotides, wherein at least one of the nucleotides is modified. The oligonucleotide hybridizes for example with a nucleic acid sequence of indoleamine-2,3-dioxygenase (IDO1) of SEQ ID NO.1 (human) and/or a sequence of SEQ ID NO.2 (mouse/rat). The modified nucleotide is for example selected from the group consisting of a bridged nucleic acid (e.g., LNA, cET, ENA, 2′Fluoro modified nucleotide, 2′O-Methyl modified nucleotide or a combination thereof). In some embodiments, the oligonucleotide inhibits at least 50% of the IDO1 expression and in some embodiments the oligonucleotide inhibits the expression of IDO1 at a nanomolar concentration.

The present invention is further directed to a pharmaceutical composition comprising an immunosuppression-reverting oligonucleotide of the present invention and optionally a pharmaceutically acceptable carrier, excipient, dilutant or a combination thereof. In some embodiments, this pharmaceutical composition additionally comprises a chemotherapeutic such as platinum or gemcitabine, another oligonucleotide, an antibody or a fragment thereof such as a Fab fragment, a HERA fusion protein, a ligand trap, a nanobody, a BiTe and/or a small molecule which is for example effective in tumor treatment.

In some embodiments, the oligonucleotide of the present invention is in combination with another oligonucleotide, an antibody and/or a small molecule, either each of these compounds is separate or combined in a pharmaceutical composition, wherein the oligonucleotide, the antibody or a fragment thereof such as a Fab fragment, a HERA fusion protein, a ligand trap, a nanobody, a BiTe and/or the small molecule inhibits or stimulates an immune suppressive factor such as IDO1, IDO2, CTLA-4, PD-1, PD-L1, LAG-3, VISTA, A2AR, CD39, CD73, STAT3, TDO2, TIM-3, TIGIT, TGF-beta, BTLA, MICA, NKG2A, KIR, CD160, Chop, and/or Xbp1. In addition or alternatively, the oligonucleotide, the antibody or a fragment thereof such as a Fab fragment, a HERA fusion protein, a ligand trap, a nanobody, a BiTe and/or the small molecule inhibits or stimulates an immune stimulatory factor such as 4-1BB, Ox40, KIR, GITR, CD27 and/or 2B4.

Furthermore, the present invention relates to the use of the oligonucleotide or the pharmaceutical composition of the present invention in a method of preventing and/or treating a disorder, where an IDO imbalance is involved. In some embodiments, the disorder is for example an autoimmune disorder, an immune disorder, a psychiatric disorder and/or cancer. In some embodiments, the oligonucleotide or the pharmaceutical composition of the present invention is for example administered locally or systemically.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### DESCRIPTION OF FIGURES

FIG. 1 shows the mRNA sequence of human (h) IDO-1 (SEQ ID No. 1; reference NM_002164.5).

FIG. 2 depicts the distribution of hIDO-1 antisense oligonucleotide binding sites on the hIDO1 mRNA of SEQ ID No. 1 as well as their modification(s) and length. hIDO1 antisense oligonucleotides were aligned to the hIDO1 mRNA sequence. The different grayscales indicate the different LNA modifications and symbols indicate the different length of the antisense oligonucleotides.

FIGS. 3A and 3B depict hIDO1 mRNA knockdown efficacy of hIDO1 antisense oligonucleotides in human cancer cell lines EFO-21 (ovarian cystadenocarcinoma;

FIG. 3A parts 1 and 2) and SKOV-3 (ovarian adenocarcinoma; FIG. 3B parts 1 and 2). EFO-21 and SKOV-3 cells were treated for 3 days with 10 μM of the respective antisense oligonucleotide. As negative control, cells were treated with neg1, an antisense oligonucleotide having the sequence CGTTTAGGCTATGTACTT (described in WO2014154843 A1). Residual hIDO1 mRNA expression relative to untreated cells is depicted. Expression values were normalized to expression of the housekeeping gene HPRT1.

FIG. 4 shows a correlation analysis of the efficacy of antisense oligonucleotides in EFO-21 and SKOV-3 cells.

FIG. 5 shows concentration-dependent hIDO1 mRNA knockdown by selected hIDO1 antisense oligonucleotides in EFO-21 cells which were A06007H (SEQ ID No.4), A06008H (SEQ ID No.11), A06030H (SEQ ID No.3), A06043H (SEQ ID No.45), A06044H (SEQ ID No.46), A06045H (SEQ ID No.47) and A06046H (SEQ ID No.48). EFO-21 cells were treated for 3 days with the indicated concentration of the respective antisense oligonucleotide. Residual hIDO1 expression is depicted compared to untreated control cells. hIDO1 mRNA expression values were normalized to expression of the housekeeping gene HPRT1. Concentration-dependent target knockdown was used for calculation of IC₅₀ values shown in Table 10.

FIG. 6A-6C depict a concentration-dependent hIDO1 mRNA- and protein knockdown by A06007H (SEQ ID No.4) and A06030H (SEQ ID No.3). Analysis of protein expression by flow cytometry (FIG. 6A), mRNA expression by QuantiGene Singleplex assay (FIG. 6B) and cell viability by cell titer blue assay (FIG. 6C) was performed in EFO-21 cells after treatment with the indicated antisense oligonucleotides for 6 days. As a control, cells were treated with neg1 for 6 days at 304. Relative expression/viability compared to untreated control cells (=1) is depicted.

FIGS. 7A and 7B depict effects of hIDO1 knockdown on L-kynurenine production in EFO-21 cells. EFO-21 cells were treated with the indicated antisense oligonucleotides A06007H (SEQ ID No.4) and A06030H (SEQ ID No.3) for 3 days at 504. Medium was replaced and supplemented and hIDO1 protein knockdown efficacy was analyzed after 24 h by flow cytometry, residual hIDO1 expression is depicted compared to untreated cells (FIG. 7A). 24 h and 48 h after medium replacement supernatants were harvested and L-kynurenine concentration was determined by ELISA (FIG. 7B). As control, cells were treated with neg1 at 504.

FIG. 8 depicts a dose dependent effect of hIDO1 antisense oligonucleotides on the production of kynurenine in EFO-21 cells. EFO-21 cells were treated with A06007H (SEQ ID No. 4) and A06030H (SEQ ID No. 3) at different concentrations (10 nM, 30 nM, 100 nM, 300 nM, 1 μM and 3 μM, respectively).

FIG. 9 shows knockdown of hIDO1 in dendritic cells, wherein human dendritic cells (DC) were generated using a 6 day protocol. During the last 3 days cells were treated with different concentrations of the hIDO1 specific antisense oligonucleotide A06030H (SEQ ID No.3) and hIDO1 protein expression was analyzed by flow cytometry. The antisense oligonucleotide neg1 was used as a control. Residual hIDO1 protein expression compared to untreated DC is shown.

FIG. 10 depicts the effect of hIDO1 knockdown in EFO-21 cells on the proliferation of T cells in coculture. EFO-21 cells were treated with A06007H (SEQ ID No. 4) and A06030H (SEQ ID No. 3) in different concentrations (10 nM, 30 nM, 100 nM, 300 nM, 1 μM and 3 μM, respectively). T cells labeled with a proliferation dye were added three days later and activated. Proliferation was analyzed on day four of the coculture.

FIGS. 11A and 11B depict hIDO1 mRNA knockdown efficacy of hIDO1 antisense oligonucleotides in human cancer cell lines EFO-21 (ovarian cystadenocarcinoma; FIG. 11A) and SKOV-3 (ovarian adenocarcinoma; FIG. 11B). EFO-21 and SKOV-3 cells were treated for 3 days with 5 μM of the respective antisense oligonucleotide. As negative control, cells were treated with S6. Residual hIDO1 mRNA expression relative to untreated cells is depicted. Expression values were normalized to expression of the housekeeping gene HPRT1.

FIGS. 12.1 to 12.3 show concentration-dependent hIDO1 mRNA knockdown by selected hIDO1 antisense oligonucleotides of a second screening round in EFO-21 cells which were A06057H (SEQ ID No.93), A06060H (SEQ ID No.96), A06062H (SEQ ID No.99), A06065H (SEQ ID No.102), A06066H (SEQ ID No.103) and A06068H (SEQ ID No.105) and the antisense oligonucleotides A06007H (SEQ ID No. 4), A06030H (SEQ ID No. 3) and A06035H (SEQ ID No. 37) of a first screening round. EFO-21 cells were treated for 3 days with the indicated concentration of the respective antisense oligonucleotide. Residual hIDO1 expression is depicted compared to untreated control cells. hIDO1 mRNA expression values were normalized to expression of the housekeeping gene HPRT1. Concentration-dependent target knockdown was used for calculation of IC₅₀ values shown in Table 16.

FIG. 13 shows the mRNA sequence of murine (m) IDO-1 (SEQ ID No. 2; reference NM_008324.2).

FIG. 14 shows the distribution of mIDO-1 antisense oligonucleotide binding sites on the mIDO-1 mRNA of SEQ ID No. 2 (NM_008324.2) as well as their modification(s) and length. mIDO1 antisense oligonucleotide sequences were aligned to the mIDO1 mRNA sequence. The different grayscales indicate the different LNA modifications and symbols indicate the different length of the antisense oligonucleotides.

FIGS. 15A and 15B show mIDO1 mRNA knockdown efficacy of mIDO1 antisense oligonucleotides in murine cancer cell lines Renca (renal adenocarcinoma; FIG. 15A) and 4T1 (mammary carcinoma; FIG. 15B). Renca and 4T1 cells were treated for 3 days with 10 μM of the respective antisense oligonucleotide. As negative control, cells were treated with neg1, an antisense oligonucleotide having the sequence CGTTTAGGCTATGTACTT. Residual mIDO1 mRNA expression relative to untreated cells is depicted. Expression values were normalized to expression of the housekeeping gene HPRT1.

FIG. 16 shows a correlation analysis of the efficacy of antisense oligonucleotides in Renca and 4T1 cells.

FIGS. 17.1 to 17.3 shows concentration-dependent mIDO1 mRNA knockdown by selected mIDO1 antisense oligonucleotides in Renca cells which were A06013MR (SEQ ID No.74), A06019MR (SEQ ID No.80), A06020MR (SEQ ID No.81), A06021MR(SEQ ID No.82), A06026MR (SEQ ID No.87), A06031MR (SEQ ID No.60) and A06032MR (SEQ ID No.61). Renca cells were treated for 3 days with the indicated concentration of the respective ASO. Residual mIDO1 expression is depicted compared to untreated control cells. mIDO1 mRNA expression values were normalized to expression of the housekeeping gene HPRT1.

FIG. 18A to 18E depicts antisense oligonucleotide-mediated in vivo mIDO1 knockdown in a syngeneic mouse tumor model. A06032MR(SEQ ID No. 61) was tested in a mouse tumor model and its administration resulted in a knockdown of IDO1 in tumor cells (FIG. 18B), monocytic myeloid-derived suppressor cells (M-MDSC) (FIG. 18C), tumor-associated macrophages (FIG. 18D) and in granulocytic myeloid-derived suppressor cells (FIG. 18E).

### DETAILED DESCRIPTION

The present invention provides for the first time human and murine oligonucleotides which hybridize with mRNA sequences of indoleamine-2,3-dioxygenase (IDO) such as IDO1 and inhibit the expression and activity, respectively, of IDO. In consequence, the level of tryptophan increases and the level of metabolites of tryptophan such as kynurenine decreases. Thus, the oligonucleotides of the present invention represent an interesting and highly efficient tool for use in a method of preventing and/or treating disorders, where the IDO such as the IDO1 expression and activity, respectively, is increased.

In the following, the elements of the present invention will be described in more detail. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

Oligonucleotides of the present invention are for example antisense oligonucleotides consisting of or comprising 10 to 25 nucleotides, 10 to 15 nucleotides, 15 to 20 nucleotides, 12 to 18 nucleotides, or 14 to 17 nucleotides. The oligonucleotides for example consist of or comprise 10, 11, 12, 13, 14, 15, 16, 17, 18, 19, 20 or 25 nucleotides. The oligonucleotides of the present invention comprise at least one nucleotide which is modified. The modified nucleotide is for example a bridged nucleotide such as a locked nucleic acid (LNA, e.g., 2′,4′-LNA), cET, ENA, a 2′Fluoro modified nucleotide, a 2′O-Methyl modified nucleotide or a combination thereof. In some embodiments, the oligonucleotide of the present invention comprises nucleotides having the same or different modifications. In some embodiments the oligonucleotide of the present invention comprises a modified phosphate backbone, wherein the phosphate is for example a phosphorothioate and/or methylphosophonate, i.e., the oligonucleotide comprise phosphorothioate or methylphosphonate or both.

The oligonucleotide of the present invention comprises the one or more modified nucleotide at the 3′- and/or 5′-end of the oligonucleotide and/or at any position within the oligonucleotide, wherein modified nucleotides follow in a row of 1, 2, 3, 4, 5, or 6 modified nucleotides, or a modified nucleotide is combined with one or more unmodified nucleotides. The following Tables 1 to 3 present embodiments of oligonucleotides comprising modified nucleotides for example LNA which are indicated by (+) and phosphorothioate (PTO) indicated by (*). The oligonucleotides consisting of or comprising the sequences of Tables 1 to 3 may comprise any other modified nucleotide and any other combination of modified and unmodified nucleotides. Oligonucleotides of Tables 1 and 2 hybridize with mRNA of human IDO1:

TABLE 1
List of antisense oligonucleotides hybridizing with human IDO1 for example
of SEQ ID No. 1; Neg1 is an antisense oligonucleotide representing a
negative control which is not hybridizing with IDO1 of SEQ ID No. 1.

| SEQ ID | | mRNA (Antisense) | Antisense Sequence 5′-3′ with PTO |
|---|---|---|---|
| No. | Name | Sequence 5′-3′ | (*) and LNA (+) |
| 3 | A06030H | AGGCGCTGTGACTTGT | +A*+G*+G*C*G*C*T*G*T*G*A*C*T*+T*+G*+T |
| 4 | A06007H | GATTGTCCAGGAGTT | +G*+A*+T*T*G*T*C*C*A*G*G*A*+G*+T*+T |
| 5 | A06001H | GGCGCTGTGACTTG | +G*+G*C*G*C*T*G*T*G*A*C*+T*+T*+G |
| 6 | A06002H | AGGCGCTGTGACTT | +A*+G*+G*C*G*C*T*G*T*G*A*C*T*+T |
| 7 | A06003H | ATGCGAAGAACACT | +A*+T*+G*C*G*A*A*G*A*A*C*+A*+C*+T |
| 8 | A06004H | TATATGCGAAGAAC | +T*+A*+T*A*T*G*C*G*A*A*G*+A*+A*+C |
| 9 | A06005H | ACTTAGTCACGATT | +A*+C*+T*T*A*G*T*C*A*C*G*+A*+T*+T |
| 10 | A06006H | GCCATTCTTGTAGTC | +G*+C*+C*A*T*T*C*T*T*G*T*A*+G*+T*+C |
| 11 | A06008H | CTCAACTCTTTCTCG | +C*+T*+C*A*A*C*T*C*T*T*T*C*+T*+C*+G |
| 12 | A06009H | GGCGCTGTGACTTGT | +G*+G*C*G*C*T*G*T*G*A*C*T*+T*+G*+T |
| 13 | A06010H | TTGGCAAGACCTTAC | +T*+T*+G*G*C*A*A*G*A*C*C*T*+T*+A*+C |
| 14 | A06011H | GTTGGCAGTAAGGAA | +G*+T*+T*G*G*C*A*G*T*A*A*G*+G*+A*+A |
| 15 | A06012H | GACACAGTCTGCATA | +G*+A*+C*A*C*A*G*T*C*T*G*C*+A*+T*+A |
| 16 | A06013HM | GTCAGGGGCTTATTA | +G*+T*+C*A*G*G*G*G*C*T*T*A*T*+T*+A |
| 17 | A06014H | GAGAACAAAACGTCC | +G*+A*G*A*A*C*A*A*A*A*C*G*+T*+C*+C |
| 18 | A06015H | AGTGTCCCGTTCTTG | +A*+G*+T*G*T*C*C*C*G*T*T*C*+T*+T*+G |
| 19 | A06016H | TATGCGAAGAACACT | +T*+A*+T*G*C*G*A*A*G*A*A*C*+A*+C*+T |
| 20 | A06017H | AGGACGTCAAAGCAC | +A*+G*+G*A*C*G*T*C*A*A*A*G*+C*+A*+C |
| 21 | A06018H | GAGCTGGTGGCATAT | +G*+A*+G*C*T*G*G*T*G*G*C*A*T*+A*+T |
| 22 | A06019H | GAGCTGGTGGCATAT | +G*+A*G*C*T*G*G*T*G*G*C*A*T*+A*+T |
| 23 | A06020HM | GACAAACTCACGGAC | +G*+A*+C*A*A*A*C*T*C*A*C*G*+G*+A*+C |
| 24 | A06021HM | GACAAACTCACGGAC | +G*+A*+C*A*A*A*C*T*C*A*C*G*G*+A*+C |
| 25 | A06022HM | GACAAACTCACGGAC | +G*+A*C*A*A*A*C*T*C*A*C*G*+G*+A*+C |
| 26 | A06023HM | TAAGCTTCCCGCAGG | +T*+A*+A*G*C*T*T*C*C*C*G*C*+A*+G*+G |
| 27 | A06024H | AGATGGTAGCTCCTC | +A*G*+A*T*G*G*T*A*G*C*T*C*+C*+T*+C |
| 28 | A06025H | GTACTTAGTCACGAT | +G*+T*+A*C*T*T*A*G*T*C*A*C*+G*+A*+T |
| 29 | A06026H | TGGCTTGCAGGAATC | +T*+G*+G*C*T*T*G*C*A*G*G*A*+A*+T*+C |
| 30 | A06027H | GTCTTCAGAGGTCTT | +G*+T*C*T*T*C*A*G*A*G*G*T*C*+T*+T |
| 31 | A06028H | CTTGTAGTCTGCTCCT | +C*+T*+T*G*T*A*G*T*C*T*G*C*T*+C*+C*+T |
| 32 | A06029H | GGCGCTGTGACTTGTG | +G*+G*C*G*C*T*G*T*G*A*C*T*T*+G*+T*+G |
| 33 | A06031H | GCAAGACCTTACGGAC | +G*+C*+A*A*G*A*C*C*T*T*A*C*G*+G*+A*+C |
| 34 | A06032H | GTTGGCAGTAAGGAAC | +G*+T*+T*G*G*C*A*G*T*A*A*G*G*A*+A*+C |
| 35 | A06033H | GAGAACAAAACGTCCA | +G*+A*+G*A*A*C*A*A*A*A*C*G*T+C*+C*+A |
| 36 | A06034H | CAGTCTCCATCACGAA | +C*+A*+G*T*C*T*C*C*A*T*C*A*C*+G*+A*+A |
| 37 | A06035H | AGTGTCCCGTTCTTGC | +A*+G*+T*G*T*C*C*C*G*T*T*C*T*+T*+G*+C |
| 38 | A06036H | AATATATGCGAAGAAC | +A*+A*+T*A*T*A*T*G*C*G*A*A*G*+A*+A*+C |
| 39 | A06037H | CAGGACGTCAAAGCAC | +C*+A*+G*G*A*C*G*T*C*A*A*A*G*+C*+A*+C |
| 40 | A06038H | TGAGCTGGTGGCATAT | +T*+G*+A*G*C*T*G*G*T*G*G*C*A*+T*+A*+T |
| 41 | A06039H | GACAAACTCACGGACT | +G*+A*C*A*A*A*C*T*C*A*C*G*G*+A*+C*+T |
| 42 | A06040HM | TTGCAGATGGTAGCTC | +T*+T*+G*C*A*G*A*T*G*G*T*A*G*+C*+T*+C |
| 43 | A06041H | GAGGTCTTTTGTATTG | +G*+A*+G*G*T*C*T*T*T*T*G*T*A*+T*+T*+G |
| 44 | A06042H | ATTCTTGTAGTCTGCTC | +A*+T*+T*C*T*T*G*T*A*G*T*C*T*G*+C*+T*+C |
| 45 | A06043H | CCAGACTCTATGAGATC | +C*+C*+A*G*A*C*T*C*T*A*T*G*A*G*+A*+T*+C |
| 46 | A06044H | GAGATGATCAATGCTGA | +G*+A*+G*A*T*G*A*T*C*A*A*T*G*C*+T*+G*+A |
| 47 | A06045H | AGGCGCTGTGACTTGTG | +A*+G*+G*C*G*C*T*G*T*G*A*C*T*T*+G*+T*+G |
| 48 | A06046H | GGTGATGCATCCCAGAA | +G*G*+T*G*A*T*G*C*A*T*C*C*C*A*+G*+A*+A |
| 49 | A06047H | GGCAAGACCTTACGGAC | +G*+G*+C*A*A*G*A*C*C*T*T*A*C*G*+G*+A*+C |
| 50 | A06048H | GTTGGCAGTAAGGAACA | +G*+T*+T*G*G*C*A*G*T*A*A*G*G*A*+A*+C*+A |
| 51 | A06049H | ACAAAACGTCCATGTTC | +A*+C*+A*A*A*A*C*G*T*C*C*A*T*G*+T*+T*+C |
| 52 | A06050H | AGTGTCCCGTTCTTGCA | +A*+G*+T*G*T*C*C*C*G*T*T*C*T*T*+G*+C*+A |
| 53 | A06051H | GAACTGAGCAGCATGTC | +G*+A*A*C*T*G*A*G*C*A*G*C*A*T*+G*T*+C |
| 54 | A06052H | GAGCTGGTGGCATATAT | +G*+A*+G*C*T*G*G*T*G*G*C*A*T*A*+T*+A*+T |
| 55 | A06053H | GTTCCTGTGAGCTGGTG | +G*+T*T*C*C*T*G*T*G*A*G*C*T*G*+G*+T*+G |
| 56 | A06054H | AGGACAAACTCACGGAC | +A*+G*+G*A*C*A*A*A*C*T*C*A*C*G*+G*+A*+C |
| 57 | A06055H | CCGCAGGCCAGCATCAC | +C*+C*G*C*A*G*G*C*C*A*G*C*A*T*C*A*+C |
| 58 | A06056H | TTGCAGATGGTAGCTCC | +T*+T*+G*C*A*G*A*T*G*G*T*A*G*C*T*+C*+C |
| 59 | Neg1 | | +C*+G*+T*T*T*A*G*G*C*T*A*T*G*T*A*+C*+T*+T |

Single-dose screens and dose-response investigations revealed the antisense oligonucleotides A06007H, A06008H, A06030H and A06035H as highly potent. To further explore their potential, 16 additional antisense oligonucleotides based on their basic sequences were designed and are shown in the following Table 2:

TABLE 2
List of antisense oligonucleotides hybridizing with human IDO1 for example
of SEQ ID No. 1; S6 is an antisense oligonucleotide representing a
negative control which is not hybridizing with IDO1 of SEQ ID No. 1.

| SEQ ID | | mRNA (Antisense) | Antisense Sequence 5′-3′ with |
|---|---|---|---|
| No. | Name | Sequence | PTO (*) and LNA (+) |
| 93 | A06057H | GCGCTGTGACTTGT | +G*+C*G*C*T*G*T*G*A*C*T*+T*+G*+T |
| 94 | A06058H | GCGCTGTGACTTGT | +T*+G*+T*C*C*C*G*T*T*C*T*+T*+G*+C |
| 95 | A06059H | GATTGTCCAGGAGTT | +G*+A*T*T*G*T*C*C*A*G*G*A*+G*+T*+T |
| 96 | A06060H | TGATTGTCCAGGAGT | +T*+G*+A*T*T*G*T*C*C*A*G*G*+A*+G*+T |
| 97 | A06061H | CTCAACTCTTTCTCG | +C*T*+C*A*A*C*T*C*T*T*T*C*+T*+C*+G |
| 99 | A06062H | AGGCGCTGTGACTTG | +A*+G*+G*C*G*C*T*G*T*G*A*C*T*+T*+G |
| 100 | A06063H | GTGTCCCGTTCTTGC | +G*+T*+G*T*C*C*C*G*T*T*C*T*+T*+G*+C |
| 101 | A06064H | GATTGTCCAGGAGTTT | +G*+A*T*T*G*T*C*C*A*G*G*A*G*+T*+T*+T |
| 102 | A06065H | TGATTGTCCAGGAGTT | +T*+G*+A*T*T*G*T*C*C*A*G*G*A*+G*+T*+T |
| 103 | A06066H | TCTCAACTCTTTCTCG | +T*+C*+T*C*A*A*C*T*C*T*T*T*C*+T*+C*+G |
| 104 | A06067H | TTCTCAACTCTTTCTC | +T*+T*+C*T*C*A*A*C*T*C*T*T*T*+C*+T*+C |
| 105 | A06068H | AGGCGCTGTGACTTGT | +A*+G*+G*C*G*C*T*G*T*G*A*C*T*T*+G*+T |
| 106 | A06069H | AGTGTCCCGTTCTTGC | +A*+G*+T*G*T*C*C*C*G*T*T*C*T*T*+G*+C |
| 107 | A06070H | GATTGTCCAGGAGTTTT | +G*+A*+T*T*G*T*C*C*A*G*G*A*G*T*+T*+T*+T |
| 108 | A06071H | CTCAACTCTTTCTCGAA | +C*+T*+C*A*A*C*T*C*T*T*T*C*T*C*+G*+A*+A |
| 109 | A06072H | CTCAACTCTTTCTCGAA | C*+T*+C*+A*A*C*T*C*T*T*T*C*T*C*+G*+A*+A |
| 110 | S6 | | +T*+C*+T*A*T*C*G*T*G*A*T*G*T*T*+T*+C*+T |

The following Table 3 shows oligonucleotides hybridizing with mRNA of rat or murine IDO1:

TABLE 3
List of antisense oligonucleotides hybridizing with rat or murine IDO1 for
example of SEQ ID No. 2; Neg1 is an antisense oligonucleotide representing
a negative control which is not hybridizing with IDO1 of SEQ ID No. 2.

| SEQ | | mRNA (Antisense) | Antisense Sequence 5′-3′ with PTO |
|---|---|---|---|
| ID No. | Name | Sequence 5′-3′ | (*) and LNA (+) |
| 60 | A06031MR | TGTATCTTTCACACTCC | +T*+G*+T*A*T*C*T*T*T*C*A*C*A*C*+T*+C*+C |
| 61 | A06032MR | GTTGTATCTTTCACACT | +G*+T*+T*G*T*A*T*C*T*T*T*C*A*C*+A*+C*+T |
| 62 | A06001MR | GGCGCTGTAACCTGT | +G*+G*+C*G*C*T*G*T*A*A*C*C*+T*+G*+T |
| 63 | A06002MR | TCGGTTCCACACATA | +T*+C*+G*G*T*T*C*C*A*C*A*C*+A*+T*+A |
| 64 | A06003MR | TCCCCTCGGTTCCAC | +T*+C*C*C*C*T*C*G*G*T*T*C*C*A*+C |
| 65 | A06004MR | GTCCATGTTCTCGTA | +G*+T*C*C*A*T*G*T*T*C*T*C*+G*+T*+A |
| 66 | A06005MR | TCGCAGTCCCCACCA | +T*C*+G*C*A*G*T*C*C*C*C*A*C*C*+A |
| 67 | A06006MR | GAGAAGCTGCGATTT | +G*+A*+G*A*A*G*C*T*G*C*G*A*+T*+T*+T |
| 68 | A06007MR | TCACGCATCCTCTTA | +T*+C*+A*C*G*C*A*T*C*C*T*C*+T*+T*+A |
| 69 | A06008MR | AAGTCACGCATCCTC | +A*+A*+G*T*C*A*C*G*C*A*T*C*+C*+T*+C |
| 70 | A06009MR | AGGCGCTGTAACCTGT | +A*G*+G*C*G*C*T*G*T*A*A*C*C*T*G*+T |
| 71 | A06010MR | TCGGTTCCACACATAC | +T*+C*+G*G*T*T*C*C*A*C*A*C*A*+T*+A*+C |
| 72 | A06011MR | CATCCCCTCGGTTCCA | +C*+A*+T*C*C*C*C*T*C*G*G*T*T*+C*+C*+A |
| 73 | A06012MR | GGCAGCACCTTTCGAA | +G*+G*+C*A*G*C*A*C*C*T*T*T*C*+G*+A*+A |
| 74 | A06013MR | GAGAGCTCGCAGTAGG | +G*+A*G*A*G*C*T*C*G*C*A*G*T*+A*+G*+G |
| 75 | A06014MR | TGTCCATGTTCTCGTA | +T*+G*+T*C*C*A*T*G*T*T*C*T*C*+G*+T*+A |
| 76 | A06015MR | TCGCAGTCCCCACCAG | +T*+C*G*C*A*G*T*C*C*C*C*A*C*C*A*+G |
| 77 | A06016MR | AAGCTGCGATTTCCAC | +A*+A*+G*C*T*G*C*G*A*T*T*T*C*+C*+A*+C |
| 78 | A06017MR | AGTCACGCATCCTCTT | +A*+G*+T*C*A*C*G*C*A*T*C*C*T*+C*+T*+T |
| 79 | A06018MR | TGACAAACTCACGGAC | +T*+G*+A*C*A*A*A*C*T*C*A*C*G*+G*+A*+C |
| 80 | A06019MR | GTTGTATCTTTCACAC | +G*+T*+T*G*T*A*T*C*T*T*T*C*A*+C*+A*+C |
| 81 | A06020MR | AGTGGATGTGGTAGAGC | +A*+G*+T*G*G*A*T*G*T*G*G*T*A*G*+A*+G*+C |
| 82 | A06021MR | AGGCGCTGTAACCTGTG | +A*+G*+G*C*G*C*T*G*T*A*A*C*C*T*+G*+T*+G |
| 83 | A06022MR | TCGGTTCCACACATACG | +T*+C*+G*G*T*T*C*C*A*C*A*C*A*T*+A*+C*+G |
| 84 | A06023MR | CCTCGGTTCCACACATA | +C*+C*+T*C*G*G*T*T*C*C*A*C*A*C*+A*+T*+A |
| 85 | A06024MR | ATGTCCATGTTCTCGTA | +A*+T*+G*T*C*C*A*T*G*T*T*C*T*C*+G*+T*+A |
| 86 | A06025MR | TCGCAGTCCCCACCAGG | +T*+C*+G*C*A*G*T*C*C*C*C*A*C*C*A*+G*+G |
| 87 | A06026MR | ATTGCTTTGATTGCAGG | +A*+T*+T*G*C*T*T*T*G*A*T*T*G*C*+A*+G*+G |
| 88 | A06027MR | GTCACGCATCCTCTTAA | +G*+T*+C*A*C*G*C*A*T*C*C*T*C*T*+T*+A*+A |
| 89 | A06028MR | AGTCACGCATCCTCTTA | +A*+G*+T*C*A*C*G*C*A*T*C*C*T*C*+T*+T*+A |
| 90 | A06029MR | GAAGGACATCAAGACTC | +G*+A*+A*G*G*A*C*A*T*C*A*A*G*A*+C*+T*+C |
| 91 | A06030MR | GCTGGAGGCATGTACTC | +G*+C*+T*G*G*A*G*G*C*A*T*G*T*A*+C*+T*+C |
| 92 | Neg1 | | +C*+G*+T*T*T*A*G*G*C*T*A*T*G*T*A*+C*+T*+T |

The oligonucleotides of the present invention hybridize for example with mRNA of human or murine IDO of SEQ ID No. 1 and/or SEQ ID No. 2. Such oligonucleotides are called IDO antisense oligonucleotides. In some embodiments, the oligonucleotides hybridize within a hybridizing active area which is one or more region(s) on the IDO mRNA, e.g., of SEQ ID NO.1, where hybridization with an oligonucleotide highly likely results in a potent knockdown of the IDO expression. In the present invention surprisingly several hybridizing active areas were identified for example selected from position 250 to 455, position 100 to 160, position 245 to 305, position 300 to 360, and/or position 650 to 710 (including the terminal figures of the ranges) of IDO1 mRNA for example of SEQ ID No. 1. Examples of antisense oligonucleotides hybridizing within the above mentioned positions of IDO1 mRNA for example of SEQ ID No. 1 are shown in the following Tables 4 to 7:

TABLE 4
Nucleotide position 100 to 160 of IDO1 mRNA of SEQ ID No. 1

| | SEQ ID |
|---|---|
| Binding site on hIDO1 mRNA | No . . . /ASO |
| (Position of the first nucleotide) | name |
| 131 | 107/A06070H |
| 132 | 101/A06064H |
| 133 | 4/A06007H |
| 133 | 95/A06059H |
| 133 | 102/A06065H |
| 134 | 96/A06060H |

TABLE 5
Nucleotide position 245 to 305 of IDO1 mRNA of SEQ ID No. 1

| | SEQ ID |
|---|---|
| Binding site on hIDO1 mRNA | No . . . /ASO |
| (Position of the first nucleotide) | name |
| 280 | 11/A06008H |
| 280 | 97/A06061H |
| 280 | 103/A06066H |
| 281 | 104/A06067H |
| 278 | 108/A06071H |
| 278 | 109/A06072H |

TABLE 6
Nucleotide position 300 to 360 of IDO1 mRNA of SEQ ID No. 1

| | SEQ ID |
|---|---|
| Binding site on hIDO1 mRNA | No . . . /ASO |
| (Position of the first nucleotide) | name |
| 332 | 3/A06030H |
| 332 | 93/A06057H |
| 332 | 94/A06058H |
| 333 | 99/A06062H |
| 332 | 105/A06068H |

TABLE 7
Nucleotide position 650 to 710 of IDO1 mRNA of SEQ ID No. 1

| | SEQ ID |
|---|---|
| Binding site on hIDO1 mRNA | No . . . /ASO |
| (Position of the first nucleotide) | name |
| 684 | 37/A06035H |
| 684 | 100/A06063H |
| 684 | 106/A06069H |

In Tables 4 to 7 “ASO” is the abbreviation for “antisense oligonucleotide” and the sequences and LNA patterns of the ASOs are specified in Tables 1 and 2.

In some embodiments, the oligonucleotide of the present invention inhibits at least about 50%, 55%, 60%, 65%, 70%, 75%, 80%, 85%, 90%, 92%, 94%, 95%, 96%, 97%, 98%, 99% or 100% of IDO such as the, e.g., human, rat or murine, IDO1 expression. Thus, the oligonucleotides of the present invention are immunosuppression-reverting oligonucleotides which revert immunosuppression for example in a cell, tissue, organ, or a subject. The oligonucleotide of the present invention inhibits the expression of IDO such as IDO1 at a nanomolar or micromolar concentration for example in a concentration of 0.1, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 950 nM, or 1, 10 or 100 μM.

In some embodiments, the oligonucleotide of the present invention is used in a concentration of 1, 3, 5, 9, 10, 15, 27, 30, 40, 50, 75, 82, 100, 250, 300, 500, or 740 nM, or 1, 2.2, 3, 5, 6.6 or 10 μM.

In some embodiments the present invention refers to a pharmaceutical composition comprising an oligonucleotide of the present invention and a pharmaceutically acceptable carrier, excipient and/or dilutant. In some embodiments, the pharmaceutical composition further comprises a chemotherapeutic, another oligonucleotide, an antibody or a fragment thereof such as a Fab fragment, a HERA fusion protein, a ligand trap, a nanobody, a BiTe and/or a small molecule.

In some embodiments, the oligonucleotide or the pharmaceutical composition of the present invention is for use in a method of preventing and/or treating a disorder. In some embodiments, the use of the oligonucleotide or the pharmaceutical composition of the present invention in a method of preventing and/or treating a disorder is combined with radiotherapy. The radiotherapy may be further combined with a chemotherapy (e.g., platinum, gemcitabine). The disorder is for example characterized by an IDO imbalance, i.e., the IDO level is increased in comparison to the level in a normal, healthy cell, tissue, organ or subject. The IDO level is for example increased by an increased IDO such as IDO1 expression and activity, respectively. The IDO level can be measured by any standard method such as immunohistochemistry, western blot, quantitative real time PCR or QuantiGene assay known to a person skilled in the art. An oligonucleotide or a pharmaceutical composition of the present invention is administered locally or systemically for example orally, sublingually, nasally, subcutaneously, intravenously, intraperitoneally, intramuscularly, intratumoral, intrathecal, transdermal, and/or rectal. Alternatively or in combination ex vivo treated immune cells are administered. The oligonucleotide is administered alone or in combination with another immunosuppression-reverting oligonucleotide of the present invention and optionally in combination with another compound such as another oligonucleotide, an antibody, a small molecule and/or a chemotherapeutic (e.g., platinum, gemcitabine). In some embodiments, the other oligonucleotide (i.e., not being part of the present invention), the antibody, and/or the small molecule are effective in preventing and/or treating an autoimmune disorder, an immune disorder, a psychiatric disorder (e.g., schizophrenia, bipolar disorders, Alzheimer's disease) and/or cancer. An oligonucleotide or a pharmaceutical composition of the present invention is used for example in a method of preventing and/or treating a solid tumor or a hematologic tumor. Examples of cancers preventable and/or treatable by use of the oligonucleotide or pharmaceutical composition of the present invention are breast cancer, lung cancer, malignant melanoma, lymphoma, skin cancer, bone cancer, prostate cancer, liver cancer, brain cancer, cancer of the larynx, gall bladder, pancreas, testicular, rectum, parathyroid, thyroid, adrenal, neural tissue, head and neck, colon, stomach, bronchi, kidneys, basal cell carcinoma, squamous cell carcinoma, metastatic skin carcinoma, osteo sarcoma, Ewing's sarcoma, reticulum cell sarcoma, liposarcoma, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilm's tumor, seminoma, ovarian tumor, leiomyomater tumor, cervical dysplasia, retinoblastoma, soft tissue sarcoma, malignant carcinoid, topical skin lesion, rhabdomyosarcoma, Kaposi's sarcoma, osteogenic sarcoma, malignant hypercalcemia, renal cell tumor, polycythermia vera, adenocarcinoma, anaplastic astrocytoma, glioblastoma multiforma, leukemia, or epidermoid carcinoma.

In some embodiments two or more oligonucleotides of the present invention are administered together, at the same time point for example in a pharmaceutical composition or separately, or on staggered intervals. In other embodiments, one or more oligonucleotides of the present invention are administered together with another compound such as another oligonucleotide (i.e., not being part of the present invention), an antibody, a small molecule and/or a chemotherapeutic, at the same time point for example in a pharmaceutical composition or separately, or on staggered intervals. In some embodiments of these combinations, the immunosuppression-reverting oligonucleotide inhibits the expression and activity, respectively, of an immune suppressive factor and the other oligonucleotide (i.e., not being part of the present invention), the antibody or a fragment thereof such as a Fab fragment, a HERA fusion protein, a ligand trap, a nanobody, a BiTe and/or small molecule inhibits (antagonist) or stimulates (agonist) the same and/or another immune suppressive factor and/or an immune stimulatory factor. The immune suppressive factor is for example selected from the group consisting of IDO1, IDO2, CTLA-4, PD-1, PD-L1, LAG-3, VISTA, A2AR, CD39, CD73, STAT3, TDO2, TIM-3, TIGIT, TGF-beta, BTLA, MICA, NKG2A, KIR, CD160, Chop, Xbp1 and a combination thereof. The immune stimulatory factor is for example selected from the group consisting of 4-1BB, Ox40, KIR, GITR, CD27, 2B4 and a combination thereof.

The immune suppressive factor is a factor whose expression and/or activity is for example increased in a cell, tissue, organ or subject. The immune stimulatory factor is a factor whose level is increased or decreased in a cell, tissue, organ or subject depending on the cell, tissue, organ or subject and its individual conditions.

An antibody in combination with the oligonucleotide or the pharmaceutical composition of the present invention is for example an anti-PD-1 antibody, an anti-PD-L1 antibody, or a bispecific antibody. A small molecule in combination with the oligonucleotide or the pharmaceutical composition of the present invention are for example NLG919, Indoximod, or Epacadostat.

A subject of the present invention is for example a mammalian, a bird or a fish.

### EXAMPLES

The following examples illustrate different embodiments of the present invention, but the invention is not limited to these examples. The following experiments are performed on cells endogenously expressing IDO1, i.e., the cells do not represent an artificial system comprising transfected reporter constructs. Such artificial systems generally show a higher degree of inhibition and lower IC50 values than endogenous systems which are closer to therapeutically relevant in vivo systems. Further, in the following experiments no transfecting agent is used, i.e., gymnotic delivery is performed. Transfecting agents are known to increase the activity of an oligonucleotide which influences the IC50 value (see for example Zhang et al., Gene Therapy, 2011, 18, 326-333; Stanton et al., Nucleic Acid Therapeutics, Vol. 22, No. 5, 2012). As artificial systems using a transfecting agent are hard or impossible to translate into therapeutic approaches and no transfection formulation has been approved so far for oligonucleotides, the following experiments are performed without any transfecting agent.

### Example 1: Design of Human IDO1 Antisense Oligonucleotides

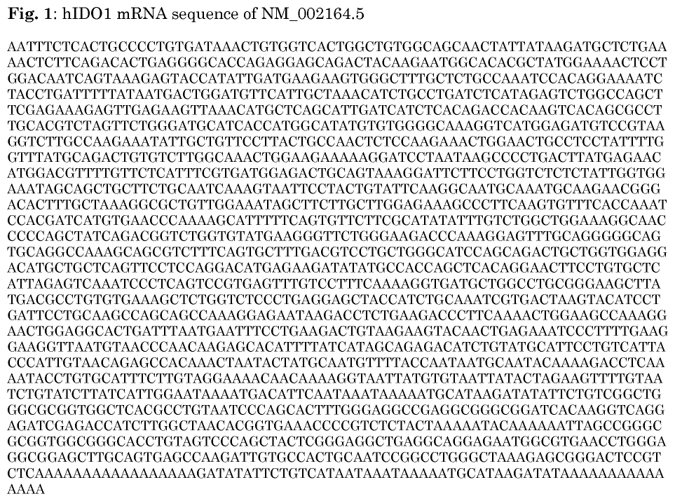

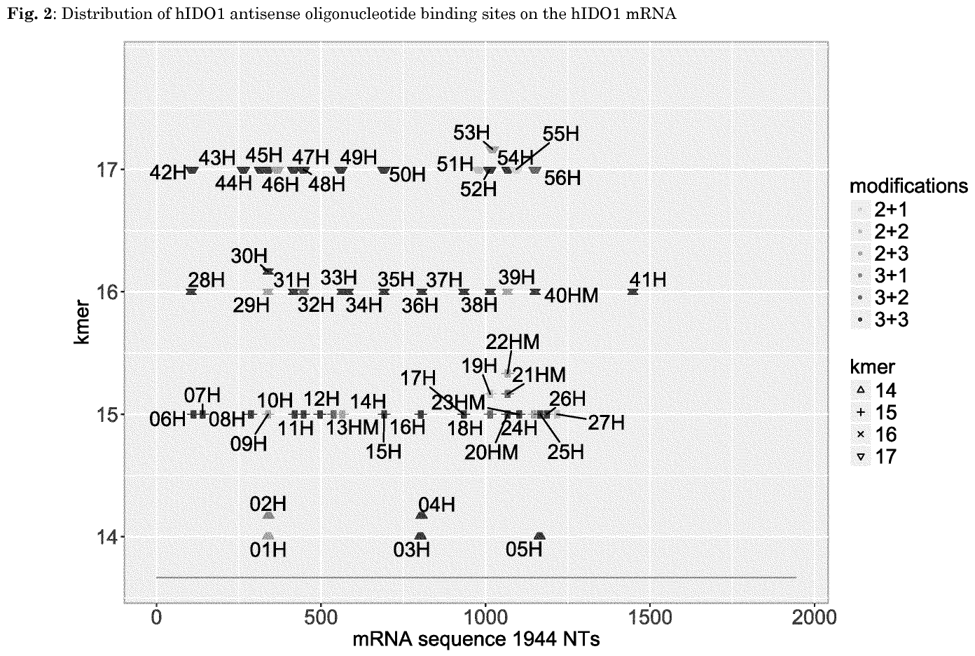

For the design of antisense oligonucleotides with specificity for human (h) IDO1 the hIDO1 mRNA sequence with SEQ ID No. 1 (seq. ref. ID NM_002164.5; FIG. 1) was used. 14, 15, 16 and 17mers were designed according to in-house criteria, neg1 (described in WO2014154843 A1) was used as control antisense oligonucleotide in all experiments (Table 1). The distribution of the antisense oligonucleotide binding sites on the hIDO1 mRNA is shown in FIG. 2.

### Example 2: Efficacy Screen of hIDO1 Antisense Oligonucleotides in Human Cancer Cell Lines

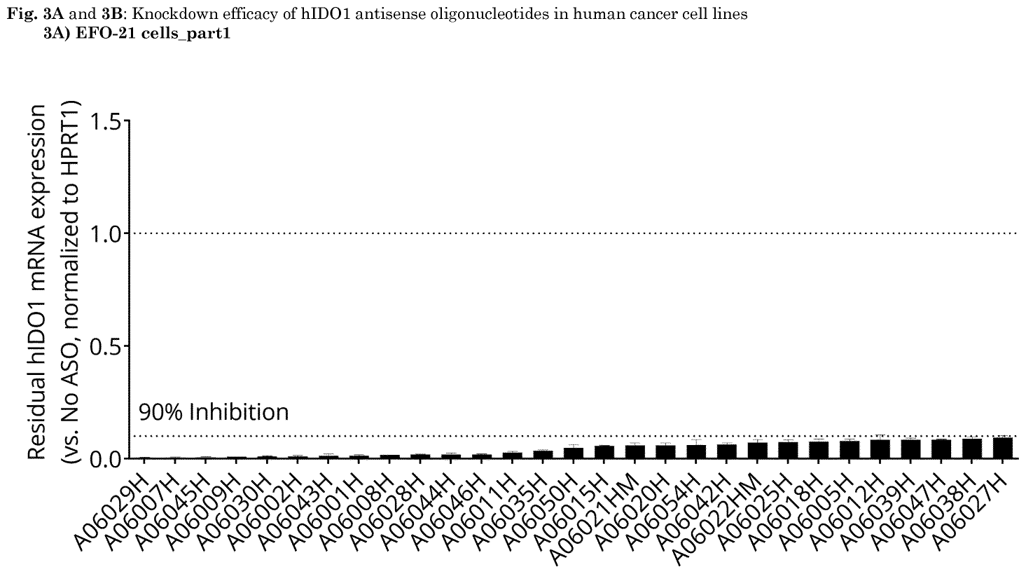

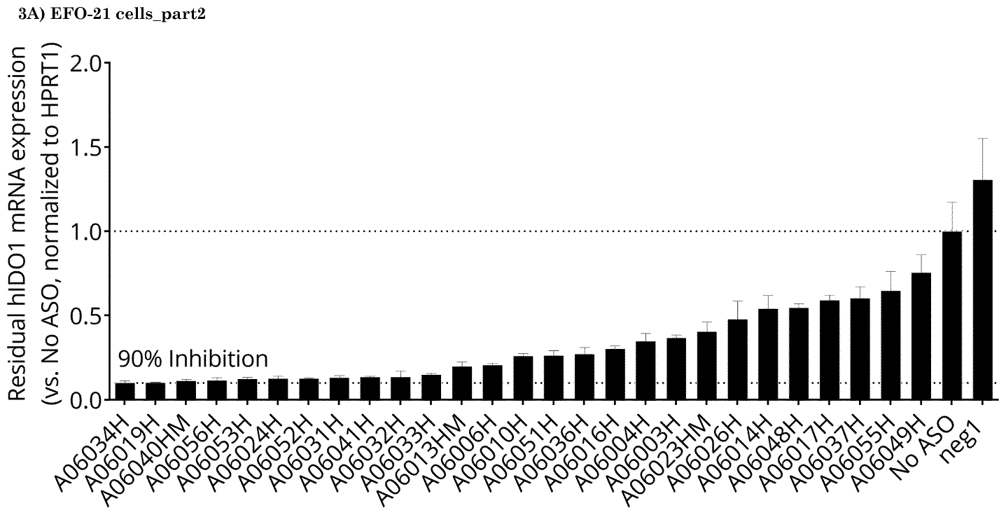

In order to analyze the efficacy of hIDO1 antisense oligonucleotides of the present invention with regard to the knockdown of hIDO1 mRNA expression in cancer cell lines, EFO-21 (human Ovarian Cystadenocarcinoma, DSMZ) and SKOV-3 (human Ovary Adenocarcinoma, ATCC) cells were treated with a single dose (concentration: 10 μM without addition of any transfection reagent; this process is called gymnotic delivery) of the respective antisense oligonucleotide as shown in FIGS. 3A and 3B. hIDO1 and HPRT1 mRNA expression was analyzed three days later using the QuantiGene Singleplex assay (Affymetrix) and hIDO1 expression values were normalized to HPRT1 values. Strikingly, as shown in FIG. 3A (EFO-21 cells) and 3B (SKOV-3 cells), a knockdown efficiency of >90% with 29 and 12 antisense oligonucleotides, respectively, was observed. Values of the mean normalized mRNA expression of hIDO1 compared to non-treated cells are listed for EFO-21 (Table 8) and SKOV-3 cells (Table 9) in the following:

TABLE 8
List of the mean normalized hIDO1 mRNA expression
values in antisense oligonucleotide-treated
EFO-21 cells compared to non-treated cells.

| | Relative hIDO1 mRNA expression |
|---|---|
| ASO | (compared to non-treated cells) |
| A06029H | 0.005 |
| A06007H | 0.006 |
| A06045H | 0.008 |
| A06009H | 0.01 |
| A06030H | 0.012 |
| A06002H | 0.012 |
| A06043H | 0.014 |
| A06001H | 0.015 |
| A06008H | 0.019 |
| A06028H | 0.019 |
| A06044H | 0.02 |
| A06046H | 0.02 |
| A06011H | 0.028 |
| A06035H | 0.037 |
| A06050H | 0.049 |
| A06015H | 0.058 |
| A06021HM | 0.061 |
| A06020H | 0.061 |
| A06054H | 0.063 |
| A06042H | 0.066 |
| A06022HM | 0.073 |
| A06025H | 0.074 |
| A06018H | 0.078 |
| A06005H | 0.08 |
| A06012H | 0.085 |
| A06039H | 0.085 |
| A06047H | 0.086 |
| A06038H | 0.089 |
| A06027H | 0.095 |
| A06034H | 0.102 |
| A06019H | 0.104 |
| A06040HM | 0.114 |
| A06056H | 0.116 |
| A06053H | 0.124 |
| A06024H | 0.127 |
| A06052H | 0.127 |
| A06031H | 0.132 |
| A06041H | 0.136 |
| A06032H | 0.136 |
| A06033H | 0.15 |
| A06013HM | 0.2 |
| A06006H | 0.207 |
| A06010H | 0.261 |
| A06051H | 0.263 |
| A06036H | 0.272 |
| A06016H | 0.303 |
| A06004H | 0.348 |
| A06003H | 0.369 |
| A06023HM | 0.406 |
| A06026H | 0.478 |
| A06014H | 0.541 |
| A06048H | 0.547 |
| A06017H | 0.592 |
| A06037H | 0.604 |
| A06055H | 0.647 |
| A06049H | 0.755 |
| Neg1 | 1.30 |

TABLE 9
List of the mean normalized hIDO1 mRNA expression
values in antisense oligonucleotide-treated
SKOV-3 cells compared to non-treated cells.

| | Relative hIDO1 mRNA expression |
|---|---|
| ASO | (compared to non-treated cells) |
| A06009H | 0.026 |
| A06029H | 0.037 |
| A06030H | 0.04 |
| A06035H | 0.041 |
| A06045H | 0.05 |
| A06001H | 0.051 |
| A06050H | 0.051 |
| A06015H | 0.054 |
| A06007H | 0.067 |
| A06028H | 0.072 |
| A06046H | 0.078 |
| A06002H | 0.091 |
| A06008H | 0.105 |
| A06044H | 0.109 |
| A06043H | 0.14 |
| A06025H | 0.143 |
| A06021HM | 0.149 |
| A06011H | 0.168 |
| A06005H | 0.178 |
| A06041H | 0.181 |
| A06020H | 0.185 |
| A06054H | 0.191 |
| A06022HM | 0.215 |
| A06038H | 0.246 |
| A06039H | 0.248 |
| A06027H | 0.251 |
| A06056H | 0.255 |
| A06033H | 0.261 |
| A06052H | 0.261 |
| A06012H | 0.262 |
| A06018H | 0.266 |
| A06042H | 0.292 |
| A06016H | 0.307 |
| A06040HM | 0.308 |
| A06019H | 0.308 |
| A06053H | 0.338 |
| A06010H | 0.357 |
| A06024H | 0.369 |
| A06034H | 0.378 |
| A06004H | 0.402 |
| A06032H | 0.402 |
| A06047H | 0.405 |
| A06003H | 0.417 |
| A06013HM | 0.443 |
| A06031H | 0.453 |
| A06026H | 0.532 |
| A06006H | 0.547 |
| A06023HM | 0.592 |
| A06051H | 0.607 |
| A06014H | 0.609 |
| A06036H | 0.659 |
| A06017H | 0.777 |
| A06048H | 0.789 |
| A06037H | 0.858 |
| A06049H | 0.89 |
| A06055H | 1.091 |
| neg1 | 1.118 |

### Example 3: Correlation Analysis of Antisense Oligonucleotide Efficacy in EFO-21 and SKOV-3 Cells

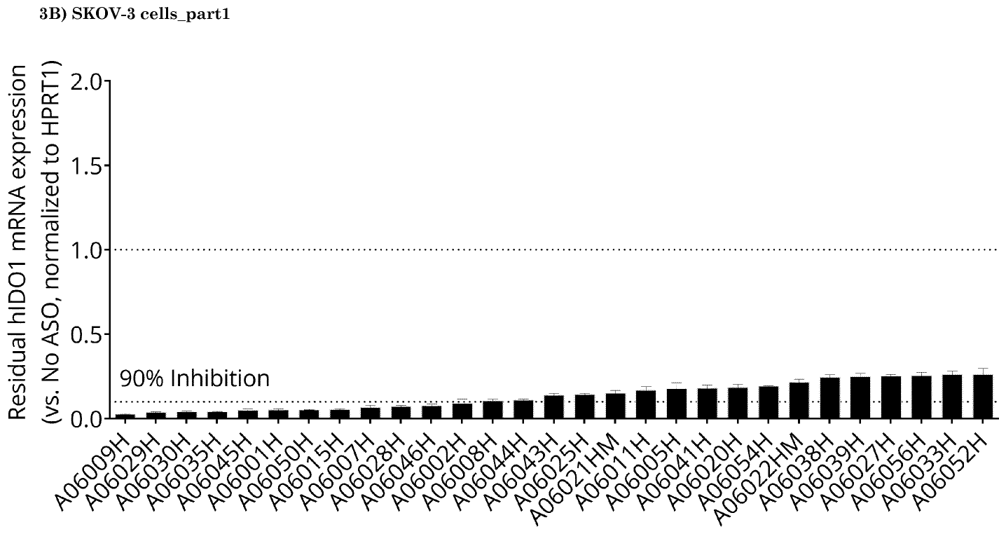

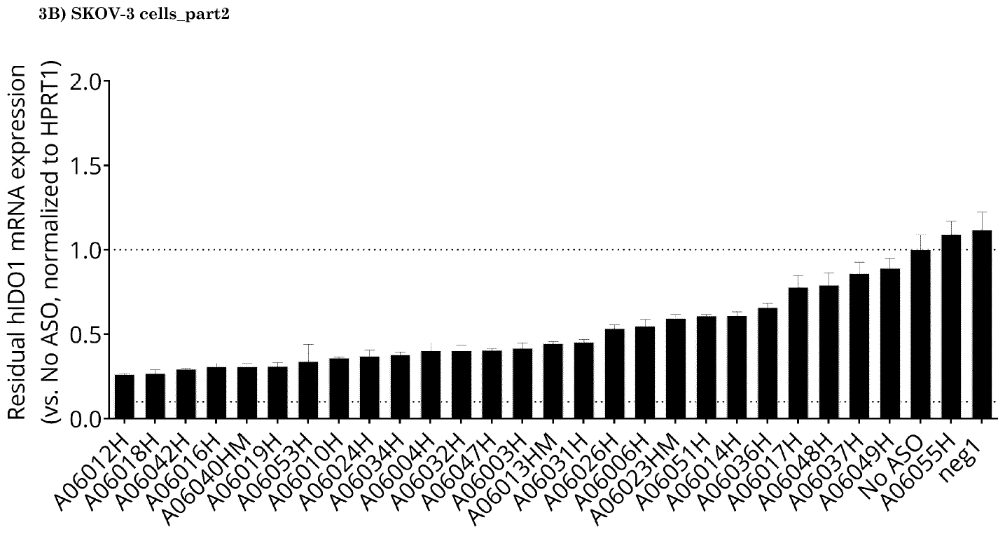

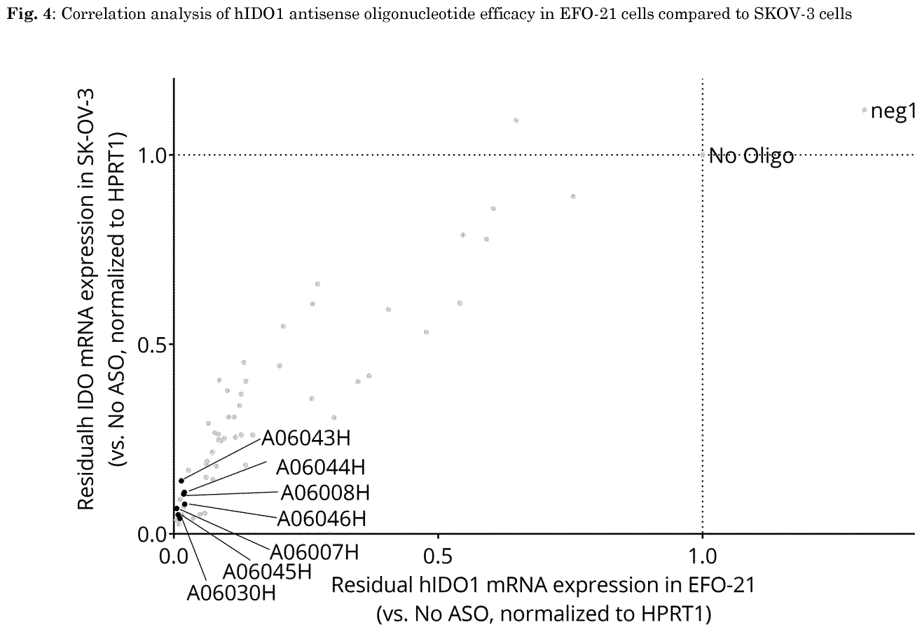

To further select the candidates with the highest activity in both tested cell lines EFO-21 and SKOV-3 a correlation analysis was performed (data derived from FIGS. 3A and 3B). As depicted in FIG. 4, 7 potent antisense oligonucleotides for determination of IC₅₀ in EFO-21 cells, namely A06007H (SEQ ID No. 4), A06008H (SEQ ID No. 11), A06030H (SEQ ID No. 3), A06043H (SEQ ID No. 45), A06044H (SEQ ID No. 46), A06045H (SEQ ID No. 47) and A06046H (SEQ ID No. 48) (marked in black) were selected. Importantly, the control antisense oligonucleotide neg1 had no negative influence on the expression of hIDO1 in both cell lines.

### Example 4: IC₅₀ Determination of Selected hIDO1 Antisense Oligonucleotides in EFO-21 Cells (mRNA Level)

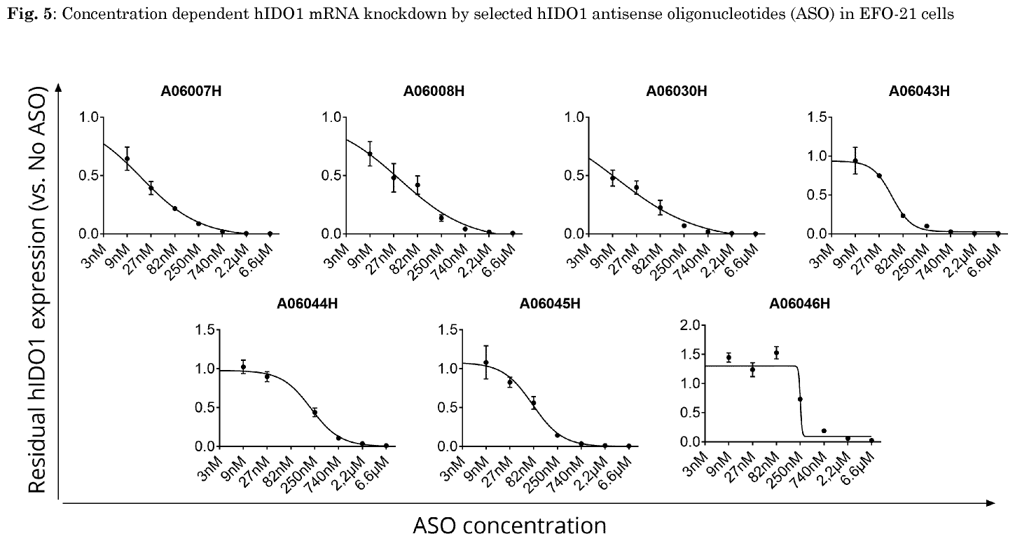

In order to determine the IC₅₀ of the hIDO1 antisense oligonucleotides A06007H (SEQ ID No. 4), A06008H (SEQ ID No. 11), A06030H (SEQ ID No. 3), A06043H (SEQ ID No. 45), A06044H (SEQ ID No. 46), A06045H (SEQ ID No. 47) and A06046H (SEQ ID No. 48), EFO-21 cells were treated with titrated amounts of the respective antisense oligonucleotide (concentrations: 6.6 μM, 2.2 μM, 740 nM, 250 nM, 82 nM, 27 nM, 9 nM, 3 nM). hIDO1 mRNA expression was analyzed three days later. As shown in FIG. 5 and following Table 10, the antisense oligonucleotides A06007H and A06030H had the highest potency in EFO-21 with regard to downregulation of hIDO1 mRNA compared to untreated cells with a maximal target inhibition of 99.7% and 99.8%, respectively. Table 10 shows IC₅₀ values and target inhibition of the above mentioned selected antisense oligonucleotides at titrated concentrations in EFO-21 cells:

TABLE 10

| | | Inhibition (%) | | | | | | | |
|---|---|---|---|---|---|---|---|---|---|
| | IC₅₀ | 6.6 | 2.2 | 740 | 250 | 82 | 27 | 9 | 3 |
| ASO | (nM) | μM | μM | nM | nM | nM | nM | nM | nM |
| A06007H | 17 | 99.7 | 99.5 | 97.9 | 91.3 | 78.3 | 60.7 | 35.6 | 26.8 |
| A06008H | 36 | 99.4 | 98.4 | 95.8 | 86.4 | 58.1 | 51.9 | 31.4 | 20.8 |
| A06030H | 11 | 99.8 | 99.5 | 98.2 | 93 | 77.4 | 60.2 | 52.2 | 36.3 |
| A06043H | 49 | 99.5 | 99.2 | 97.2 | 90 | 76.7 | 25.2 | 5.9 | 15.4 |
| A06044H | 205 | 98.9 | 96.3 | 89.2 | 56.1 | 0 | 10.2 | 0 | 7.3 |
| A06045H | 78 | 99.3 | 98.9 | 96.5 | 85.6 | 44 | 17.5 | 0 | 0 |
| A06046H | ~246 | 97.6 | 94.1 | 81 | 26.9 | 0 | 0 | 0 | 0 |

### Example 5: Detailed Characterization of Antisense Oligonucleotides A06007H and A06030H

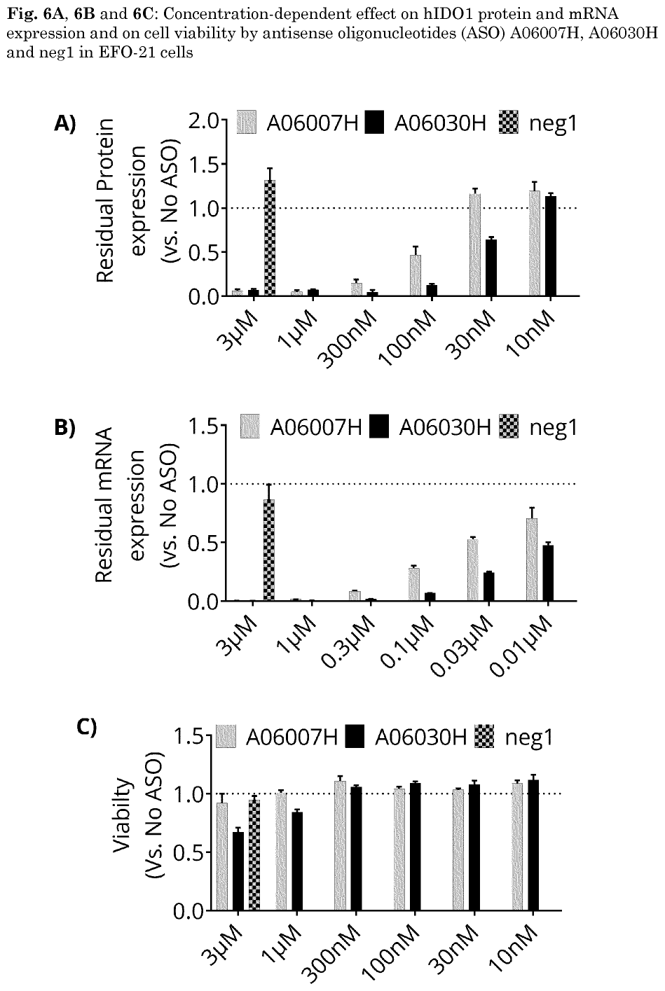

The highly potent hIDO1 antisense oligonucleotides A06007H (SEQ ID No. 4) and A06030H (SEQ ID No. 3) were characterized in detail with regard to their knockdown efficacy on the hIDO1 protein and mRNA expression and their influence on cell viability at different concentrations. EFO-21 cell were therefore treated with different concentrations of the respective antisense oligonucleotide for three days, then splitted at a ratio of 1:3 and treated again with the respective antisense oligonucleotide at the indicated concentration. After another three days, protein expression was analyzed by flow cytometry using the hIDO1 antibody clone “eyedio”, mRNA expression was analyzed and cell viability was investigated using the CellTiter-Blue Cell Viability Assay (Promega). As shown in FIG. 6A, 6B, 6C and Table 11, both antisense oligonucleotides show potent inhibition of hIDO1 protein (FIG. 6A) and mRNA expression (FIG. 6B) after 6 days antisense oligonucleotide treatment in total whereas treatment with neg1 had no inhibitory effect. Furthermore, cell viability was only mildly affected, when cells were treated with 3 μM and 1 μM of A06030H, respectively (FIG. 6C). All other conditions had no influence on the viability of EFO-21 cells. Table 11 summarizes IC₅₀ values and target inhibition on protein and mRNA level in EFO-21 cells:

TABLE 11
Overview of IC₅₀ values of hIDO1 antisense oligonucleotides

| | | Inhibition (%) (Protein/mRNA) | | | | | |
|---|---|---|---|---|---|---|---|
| | IC₅₀ (nM) | | | | | | |
| ASO | (Protein/mRNA) | 3 μM | 1 μM | 300 nM | 100 nM | 30 nM | 10 nM |
| A06007H | 80/34 | 94/99.6 | 95.2/98.5 | 85.3/91.6 | 53.4/71.9 | 0/47.3 | 0/29.4 |
| A06030H | 30/9 | 93/99.7 | 92.7/99.5 | 95.4/98.3 | 87.6/93.2 | 35.8/75.6 | 0/52.6 |

### Example 6: Downstream Effect of hIDO1 Knockdown on Kynurenine Production in EFO-21 Cells

Kynurenines (L-kynurenine, kynurenic acid, 3-hydroxykynurenine) are the major immunosuppressive molecules that are generated during tryptophan degradation by hIDO1. The first kynurenine that is produced during tryptophan degradation is L-kynurenine which can be detected in cell culture supernatants by an enzyme linked immunosorbent assay (ELISA) (L-Kynurenine ELISA kit, ImmuSmol). EFO-21 cells were treated for three days with the antisense oligonucleotides A06007H (SEQ ID No. 4) and A06030H (SEQ ID No. 3) at 504. Medium was changed to RPMI-1640 and supplemented with fresh antisense oligonucleotide at the respective concentration. As RPMI-1640 has a defined tryptophan concentration of only 24.5 μM (according to sigmaaldrich.com) RPMI-1640 was supplemented with 200 μM L-tryptophan (L-trp) in an additional experimental condition.

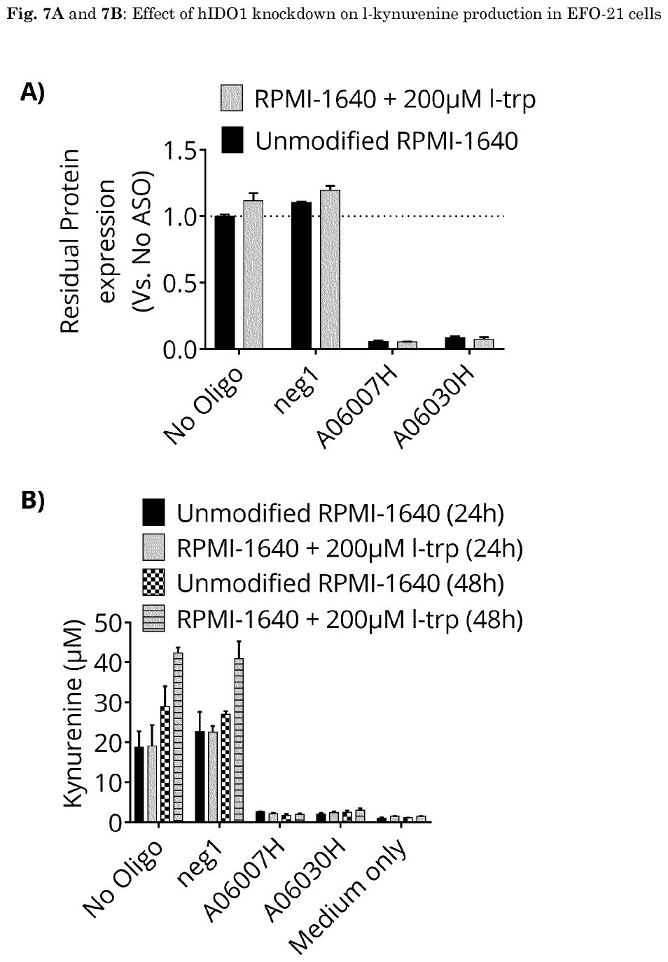

Protein knockdown efficiency of both antisense oligonucleotides was verified after 24 hours (FIG. 7A, % target inhibition: A06007H: 94.3, A06030H: 91.4), the supernatants were harvested 24 and 48 hours after medium change and L-kynurenine concentrations were analyzed by ELISA. Strikingly, L-kynurenine production was nearly completely abolished when EFO-21 were treated with A06007H (SEQ ID No. 4) or A06030H (SEQ ID No. 3) (FIG. 7B, Table 12). In contrast, treatment with the control antisense oligonucleotide neg1 had no effect on L-kynurenine production. The addition of L-tryptophan to the medium resulted in an increased kynurenine production only after 48 hours compared to unmodified RPMI-1640 in untreated and neg1 treated EFO-21 cells. Table 12 presents the effect of hIDO1 knockdown on L-kynurenine production in EFO-21 cells:

TABLE 12
Determination of L-kynurenine concentration in
supernatants of EFO-21 cells after hIDO1 protein
knockdown and after addition of L-tryptophan

L-kynurenine (μM) (24 h/48)

| | Unmodified | |
|---|---|---|
| ASO | RPMI-1640 | RPMI-1640 + l-trp |
| No ASO | 18.8/28.9 | 19.1/42.3 |
| neg1 | 22.7/27 | 22.5/40.9 |
| A06007H | 2.6/1.7 | 2.1/2 |
| A06030H | 2/2.4 | 2.3/2.9 |
| Medium | 1/1.1 | 1.5/1.5 |
| control | | |

### Example 7: Dose Dependent hIDO1 Knockdown on Kynurenine Production in EFO-21 Cells

In addition to the experiments described in Example 6, the effect of treatment of EFO-21 cells with hIDO1 antisense oligonucleotides, e.g., A06007H (SEQ ID No. 4) and A06030H (SEQ ID No. 3) at different concentrations was investigated. Therefore, EFO-21 cells were treated with 10 nM, 30 nM, 100 nM, 300 nM, 1 μM or 3 μM of the respective antisense oligonucleotide for three days. S6 was used as control antisense oligonucleotide (ASO). Medium was then changed to RPMI-1640 and supplemented with fresh antisense oligonucleotide at the respective concentration and 100 μM L-tryptophan. Supernatant was harvested 24 h later and L-kynurenine levels were determined by ELISA.

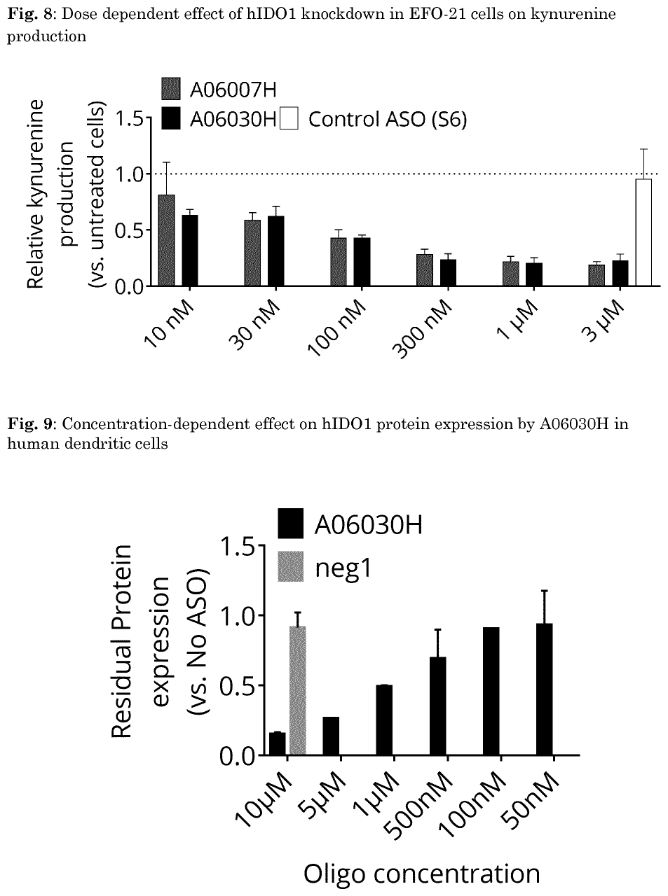

Strikingly, a potent reduction of 1-Kynurenine levels upon treatment of cells with both tested hIDO1 antisense oligonucleotides with a >50% reduction at concentrations as low as 100 nM compared to untreated cells was observed (FIG. 8).

### Example 8: Efficient Knockdown of hIDO1 in Dendritic Cells

Monocytes were enriched from peripheral blood mononuclear cells by plastic adherence. Monocytes were differentiated into dendritic cells (DC) for three days, followed by maturation for three days. DC were treated with neg1 or antisense oligonucleotide A06030H at different concentrations during the maturation period. As shown in FIG. 9 and Table 13, hIDO1 could efficiently be knocked down on the protein level with an IC₅₀ value of 1.204. Table 9 shows knockdown of hIDO1 in dendritic cells using the antisense oligonucleotide A06030H:

TABLE 13

| | | Inhibition (%) | | | | | |
|---|---|---|---|---|---|---|---|
| | IC₅₀ | 10 | 5 | 1 | 500 | 100 | 50 |
| ASO | (μM) | μM | μM | μM | nM | nM | nM |
| A06030H | 1.2 | 84.8 | 73.6 | 50.7 | 30.9 | 9.6 | 6.9 |

### Example 9: Effect of hIDO1 Knockdown in EFO-21 Cells on the Proliferation of T Cells in Coculture

Tryptophan starvation and the presence of kynurenines in the tumor microenvironment play an important role in the suppression of immune effector cells (e.g. T cells). The effect of hIDO knockdown in tumor cells on the proliferation of T cells is investigated in coculture in vitro. EFO-21 cells were treated with different concentrations of the respective antisense oligonucleotide, e.g., A06007H (SEQ ID No. 4) and A06030H (SEQ ID No. 3), respectively. S6 was used as control antisense oligonucleotide (ASO). T cells labeled with a proliferation dye were added three days later, activated with CD2/CD3/CD28 antibodies and proliferation was analyzed by flow cytometry four days after T cell activation.

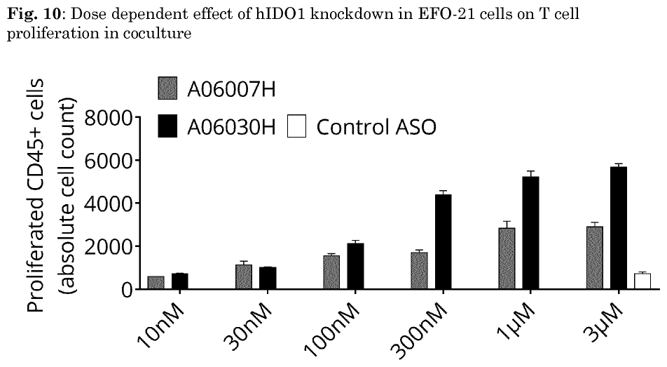

Strikingly, upon knockdown of hIDO1 in EFO-21 cells, strong proliferation of activated CD45+ cells in a concentration dependent manner was observed (FIG. 10). The strongest effect was observed upon treatment with the hIDO1 antisense oligonucleotide A06030H at a concentration of 3 μM that resulted in a 7,8 fold increased proliferation of CD45+ cells compared to the control antisense oligonucleotide condition.

### Example 10: Efficacy Screens of hIDO1 Antisense Oligonucleotides in EFO-21 and SKOV-3 Cells

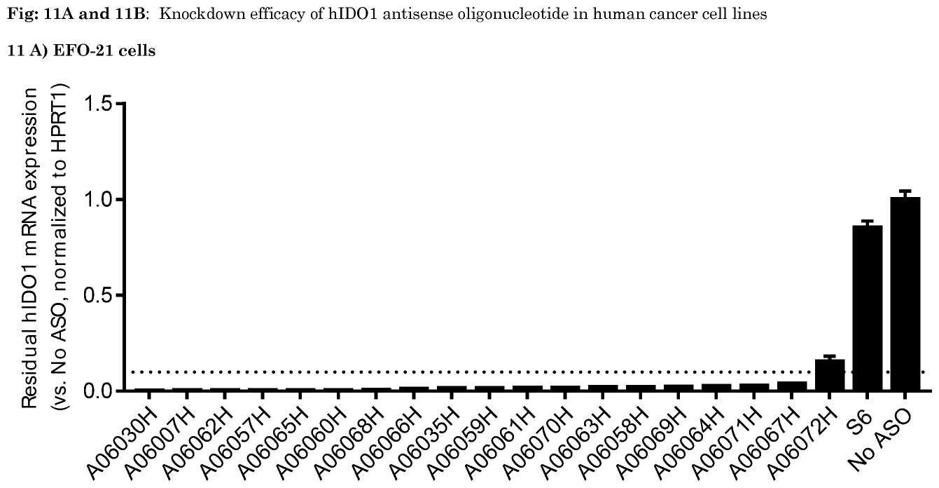

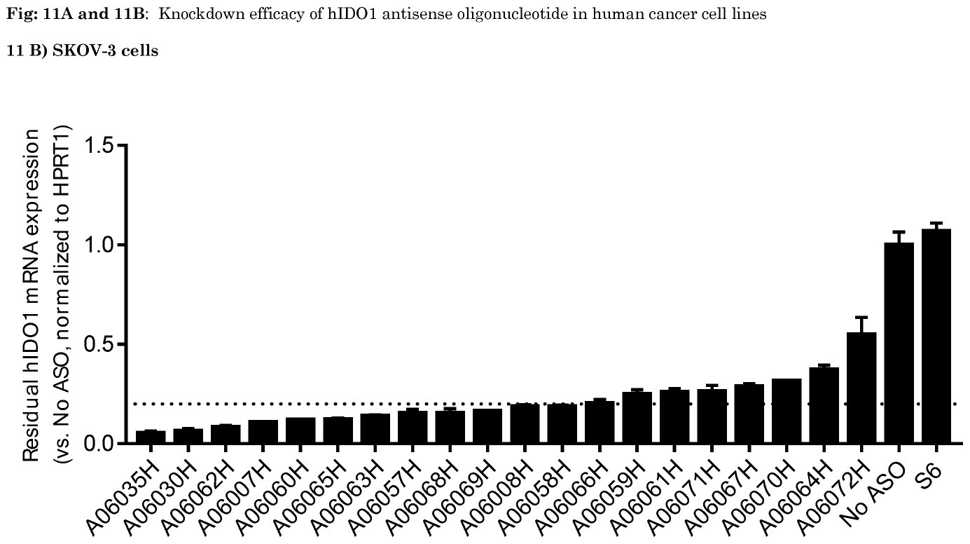

The efficacy of additional hIDO1 antisense oligonucleotides with regard to the knockdown of hIDO1 mRNA expression in cancer cell lines was investigated in a further screening round. EFO-21 (human Ovarian Cystadenocarcinoma, DSMZ) and SKOV-3 (human Ovary Adenocarcinoma, ATCC) cells were treated with the respective antisense oligonucleotide at a single dose (concentration: 504) without addition of any transfection reagent (this process is called gymnotic delivery). hIDO1 and HPRT1 mRNA expression was analyzed after three days of treatment using the QuantiGene Singleplex assay (Affymetrix) hIDO1 expression values were normalized to HPRT1 values and are shown in FIGS. 11A and 11B relative to untreated cells (set as 1). Surprisingly, a knockdown efficiency of >90% was observed in EFO-21 cells with 15 of 16 newly designed antisense oligonucleotides and all three tested antisense oligonucleotides from the first screening round, namely A06007H (SEQ ID No. 4), A06030H (SEQ ID No. 3) and A06035H (SEQ ID No. 37) (FIG. 11A). Furthermore, an efficiency of >80% was observed in SKOV-3 cells with 8 of 16 newly designed antisense oligonucleotides and all four tested antisense oligonucleotides from the first screening round, namely A06007H (SEQ ID No. 4), A06008H (SEQ ID No. 11), A06030H (SEQ ID No. 3) and A06035H (SEQ ID No. 37) (FIG. 11B). Values of the mean normalized mRNA expression of hIDO1 compared to non-treated cells (set as 1) are listed for EFO-21 (Table 14) and SKOV-3 cells (Table 15) in the following:

TABLE 14
List of mean normalized hIDO1 mRNA expression
values in antisense oligonucleotide-treated
EFO-21 cells compared to non-treated cells.

| | Relative hIDO1 mRNA expression |
|---|---|
| ASO | (compared to non-treated cells (set as 1)) |
| A06030H | 0.002 |
| A06007H | 0.003 |
| A06062H | 0.003 |
| A06057H | 0.004 |
| A06065H | 0.004 |
| A06060H | 0.005 |
| A06068H | 0.005 |
| A06066H | 0.011 |
| A06035H | 0.015 |
| A06059H | 0.015 |
| A06061H | 0.016 |
| A06070H | 0.016 |
| A06063H | 0.019 |
| A06058H | 0.021 |
| A06069H | 0.023 |
| A06064H | 0.026 |
| A06071H | 0.028 |
| A06067H | 0.039 |
| A06072H | 0.153 |
| S6 | 0.854 |

TABLE 15
List of mean normalized hIDO1 mRNA expression
values in antisense oligonucleotide-treated
SKOV-3 cells compared to non-treated cells.

| | Relative hIDO1 mRNA expression |
|---|---|
| ASO | (compared to non-treated cells (set as 1)) |
| A06035H | 0.054 |
| A06030H | 0.063 |
| A06062H | 0.084 |
| A06007H | 0.108 |
| A06060H | 0.120 |
| A06065H | 0.122 |
| A06063H | 0.139 |
| A06057H | 0.154 |
| A06068H | 0.155 |
| A06069H | 0.163 |
| A06008H | 0.187 |
| A06058H | 0.187 |
| A06066H | 0.203 |
| A06059H | 0.249 |
| A06061H | 0.259 |
| A06071H | 0.263 |
| A06067H | 0.287 |
| A06070H | 0.317 |
| A06064H | 0.372 |
| A06072H | 0.550 |
| S6 | 1.069 |

### Example 11: IC₅₀ Determination of Selected hIDO1 Antisense Oligonucleotides in EFO-21 Cells (mRNA Level)

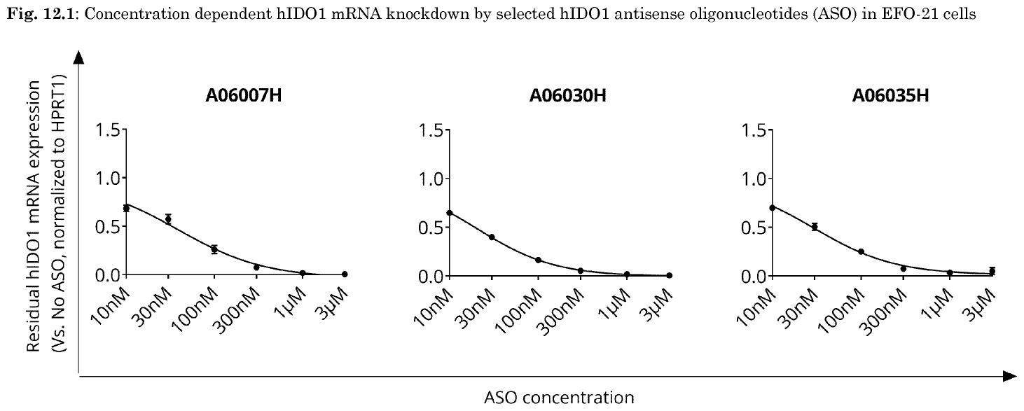

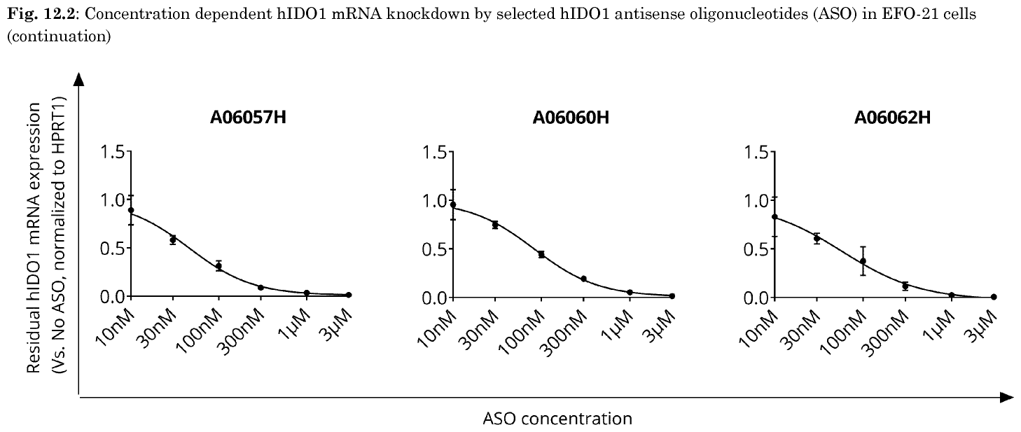

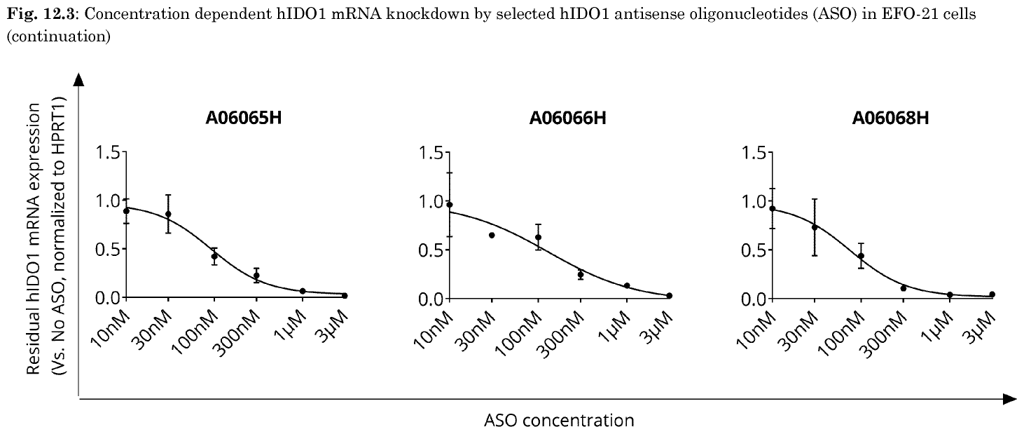

In order to determine the IC₅₀ of the potent hIDO1 antisense oligonucleotides A06057H (SEQ ID No. 99), A06060H (SEQ ID No. 96), A06062H (SEQ ID No. 99), A06065H (SEQ ID No. 102), A06066H (SEQ ID No. 103) and A06068H (SEQ ID No. 105) that have been identified in the second screening round and the antisense oligonucleotides A06007H (SEQ ID No. 4), A06030H (SEQ ID No. 3) and A06035H (SEQ ID No. 37) that have been identified in the first screening round, EFO-21 cells were treated with different concentrations of the respective antisense oligonucleotides (concentrations: 304, 1 μM, 300 nM, 100 nM, 30 nM, 10 nM). hIDO1 mRNA expression was analyzed after three days of treatment. As shown in FIG. 12 and following Table 16 all tested antisense oligonucleotides had a high potency in EFO-21 cells with regard to downregulation of hIDO1 mRNA with a maximal target inhibition between 95.0% and 99.5% compared to untreated cells. Table 16 shows IC₅₀ values and target inhibition of the above mentioned selected antisense oligonucleotides at titrated concentrations in EFO-21 cells:

TABLE 16
Overview of IC₅₀ values of hIDO1 antisense
oligonucleotides in EFO-21 cells.

| | | Inhibition (%) | | | | | |
|---|---|---|---|---|---|---|---|
| | IC₅₀ | 3 | 1 | 300 | 100 | 30 | 10 |
| ASO | (nM) | μM | μM | nM | nM | nM | nM |
| A06007H | 39 | 99.4 | 98.2 | 92.5 | 74.1 | 42.7 | 31.5 |
| A06030H | 21 | 99.5 | 98.2 | 94.7 | 83.6 | 60.2 | 35.3 |
| A06035H | 30 | 95.0 | 96.6 | 92.7 | 74.9 | 49.6 | 30.2 |
| A06057H | 46 | 98.7 | 96.6 | 91.1 | 68.5 | 42.0 | 11.0 |
| A06060H | 83 | 98.5 | 94.7 | 80.8 | 56.0 | 25.3 | 4.5 |
| A06062H | 58 | 99.4 | 97.6 | 88.5 | 62.5 | 39.4 | 17.0 |
| A06065H | 92 | 98.3 | 93.5 | 77.5 | 57.9 | 14.2 | 11.2 |
| A06066H | 129 | 97.0 | 86.6 | 75.4 | 37.2 | 35.1 | 4.0 |
| A06068H | 75 | 95.7 | 96.1 | 89.6 | 56.2 | 27.1 | 7.9 |

### Example 12: Design of Mouse/Rat IDO1 Antisense Oligonucleotides

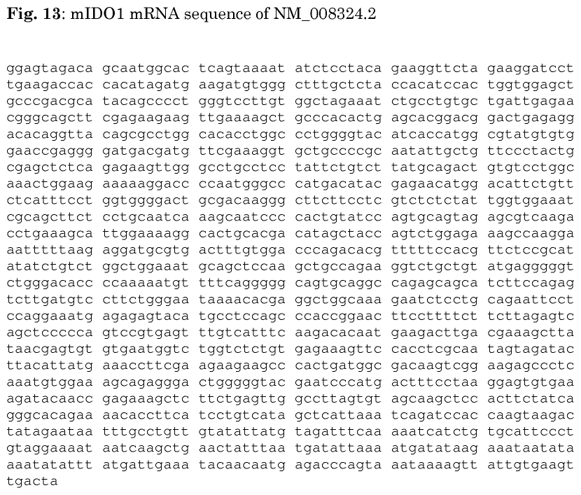

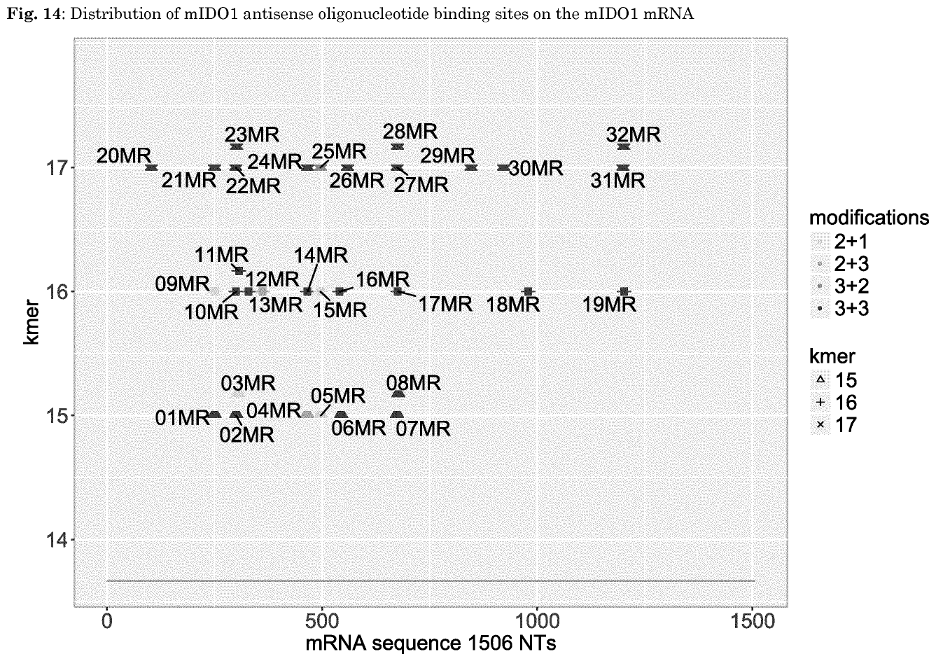

Due to the sequence differences between human and mouse(m)/rat(r) IDO1 only few hIDO1 antisense oligonucleotides are cross-reactive to mouse/rat IDO1. As they showed only limited knockdown efficacy in human cell lines, surrogate antisense oligonucleotides were designed with specificity for mouse/rat IDO1. The mouse IDO1 mRNA sequence with SEQ ID No. 2 (seq. ref. NM_008324; FIG. 13) was used as basis for the design of 15, 16 and 17mer antisense oligonucleotides, neg1 (described in WO2014154843 A1) served as control in all experiments (Table 3). The distribution of the antisense oligonucleotide binding sites on the mIDO1 mRNA is shown in FIG. 14.

### Example 13: Efficacy Screen of mIDO1 Antisense Oligonucleotides in Murine Cancer Cell Lines

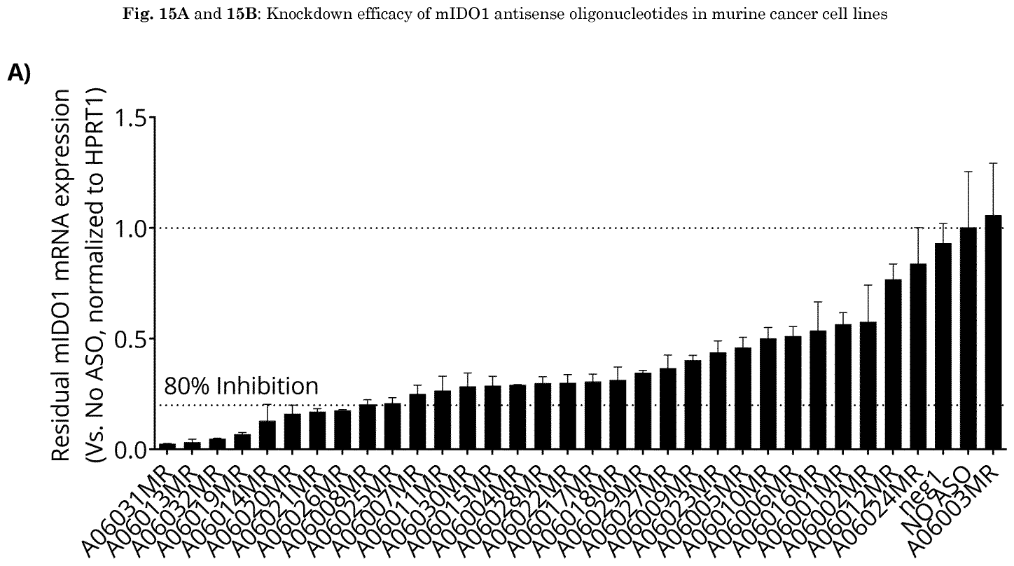

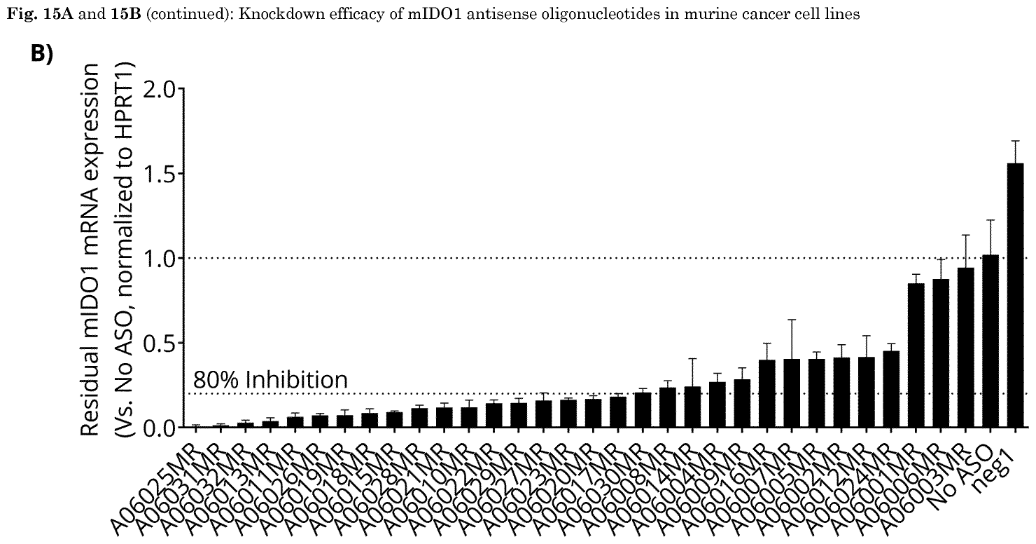

In order to analyze the efficacy of mIDO1 antisense oligonucleotides with regard to the knockdown of mIDO1 mRNA expression in cancer cell lines, Renca (mouse renal adenocarcinoma, ATCC) and 4T1 cells (tumor of the mammary gland, ATCC) cells were treated with murine interferon gamma (mIFNg) to induce mIDO1 expression and a single dose (concentration: 5 μM without addition of any transfection reagent; this process is called gymnotic delivery) of the respective antisense oligonucleotide as indicated in FIGS. 15A and 15B. mIDO1 and HPRT1 mRNA expression was analyzed three days later using the QuantiGene Singleplex assay (Affymetrix) and mIDO1 expression values were normalized to HPRT1 values. Strikingly, as shown in FIGS. 15A and 15B, treatment with 8 and 18 antisense oligonucleotides resulted in a knockdown efficacy of >80% in Renca (FIG. 15A) and 4T1 (FIG. 15B) cells, respectively. Values of the mean normalized mRNA expression of mIDO1 compared to non-treated cells are listed for Renca (Table 17) and 4T1 cells (Table 18) in the following:

TABLE 17
List of mean normalized mIDO1 mRNA expression
values in antisense oligonucleotide-treated
Renca cells compared to non-treated cells

| | Relative mIDO1 mRNA expression |
|---|---|
| ASO | (compared to non-treated cells) |
| A06031MR | 0.026 |
| A06013MR | 0.033 |
| A06032MR | 0.049 |
| A06019MR | 0.069 |
| A06014MR | 0.13 |
| A06020MR | 0.162 |
| A06021MR | 0.17 |
| A06026MR | 0.177 |
| A06008MR | 0.204 |
| A06025MR | 0.209 |
| A06007MR | 0.252 |
| A06011MR | 0.266 |
| A06030MR | 0.285 |
| A06015MR | 0.289 |
| A06004MR | 0.292 |
| A06028MR | 0.3 |
| A06022MR | 0.301 |
| A06017MR | 0.307 |
| A06018MR | 0.314 |
| A06029MR | 0.347 |
| A06027MR | 0.368 |
| A06009MR | 0.403 |
| A06023MR | 0.439 |
| A06005MR | 0.461 |
| A06010MR | 0.503 |
| A06006MR | 0.513 |
| A06016MR | 0.537 |
| A06001MR | 0.566 |
| A06002MR | 0.576 |
| A06012MR | 0.769 |
| A06024MR | 0.839 |
| neg1 | 0.931 |
| A06003MR | 1.058 |

TABLE 18
List of mean normalized mIDO1 mRNA expression values in antisense
oligonucleotide-treated 4T1 cells compared to non-treated cells

| | Relative mIDO1 mRNA expression |
|---|---|
| ASO | (compared to non-treated cells) |
| A06025MR | 0.004 |
| A06031MR | 0.014 |
| A06032MR | 0.028 |
| A06013MR | 0.038 |
| A06011MR | 0.063 |
| A06026MR | 0.071 |
| A06019MR | 0.072 |
| A06018MR | 0.086 |
| A06015MR | 0.091 |
| A06028MR | 0.115 |
| A06021MR | 0.118 |
| A06010MR | 0.119 |
| A06022MR | 0.143 |
| A06029MR | 0.145 |
| A06027MR | 0.159 |
| A06023MR | 0.164 |
| A06020MR | 0.169 |
| A06017MR | 0.181 |
| A06030MR | 0.207 |
| A06008MR | 0.237 |
| A06014MR | 0.242 |
| A06004MR | 0.269 |
| A06009MR | 0.286 |
| A06016MR | 0.399 |
| A06007MR | 0.404 |
| A06005MR | 0.405 |
| A06002MR | 0.414 |
| A06012MR | 0.416 |
| A06024MR | 0.452 |
| A06001MR | 0.851 |
| A06006MR | 0.875 |
| A06003MR | 0.943 |
| neg1 | 1.56 |

### Example 14: Knockdown Efficacy of mIDO1 Antisense Oligonucleotides in Murine Cancer Cell Lines

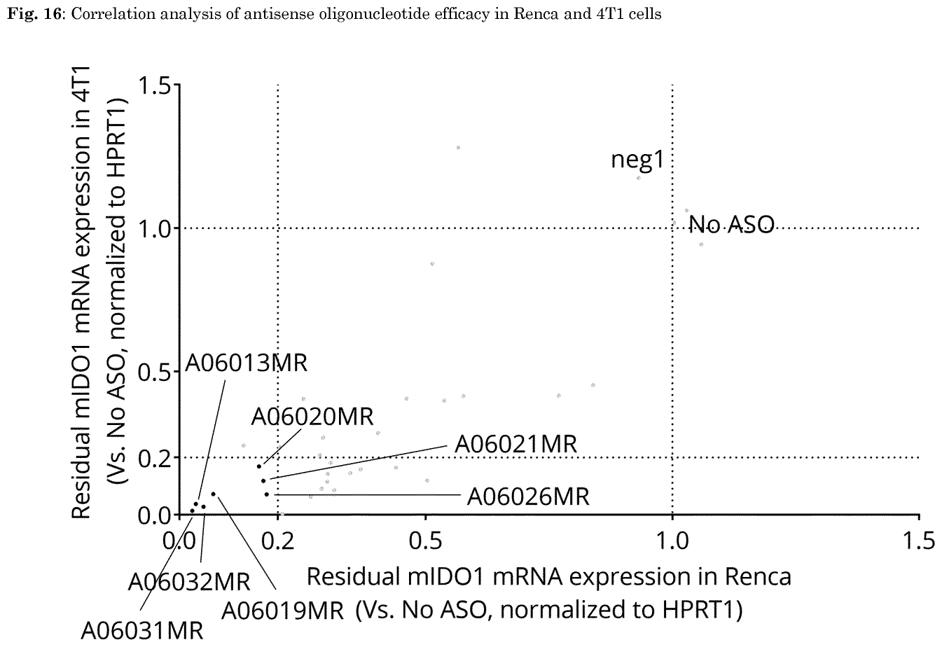

To further select the candidates with the highest activity in both tested cell lines a correlation analysis was performed (data derived from FIG. 15). As depicted in FIG. 16 potent antisense oligonucleotides were selected for determination of IC₅₀ in Renca cells, namely A06013MR (SEQ ID No. 74), A06019MR (SEQ ID No. 80), A06020MR (SEQ ID No. 81), A06021MR (SEQ ID No. 82), A06026MR (SEQ ID No. 87), A06031MR (SEQ ID No. 60) and A06032MR (SEQ ID No. 61) (marked in black). Importantly, the control antisense oligonucleotide neg1 had no negative influence on the expression of mIDO1 in both cell lines.

### Example 15: IC₅₀ Determination of Selected mIDO1 Antisense Oligonucleotides in Renca Cells (mRNA Level)

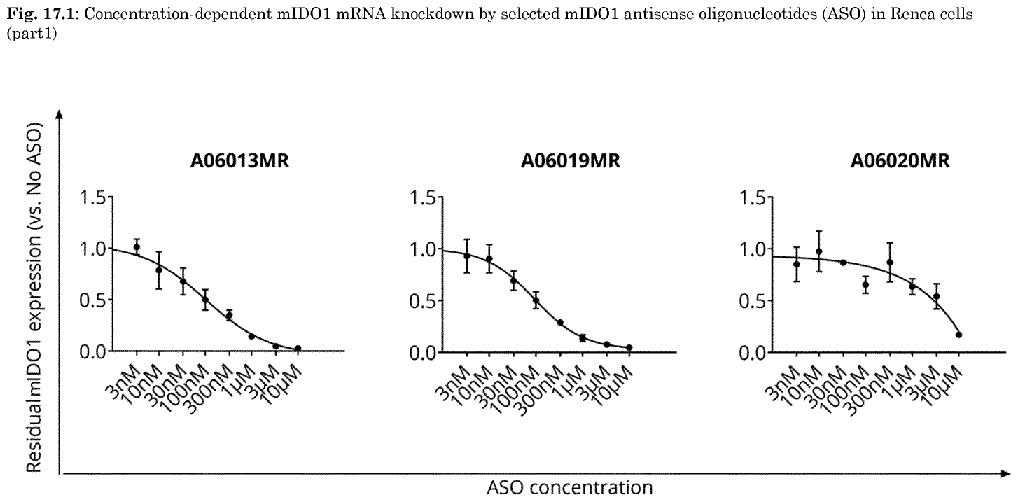

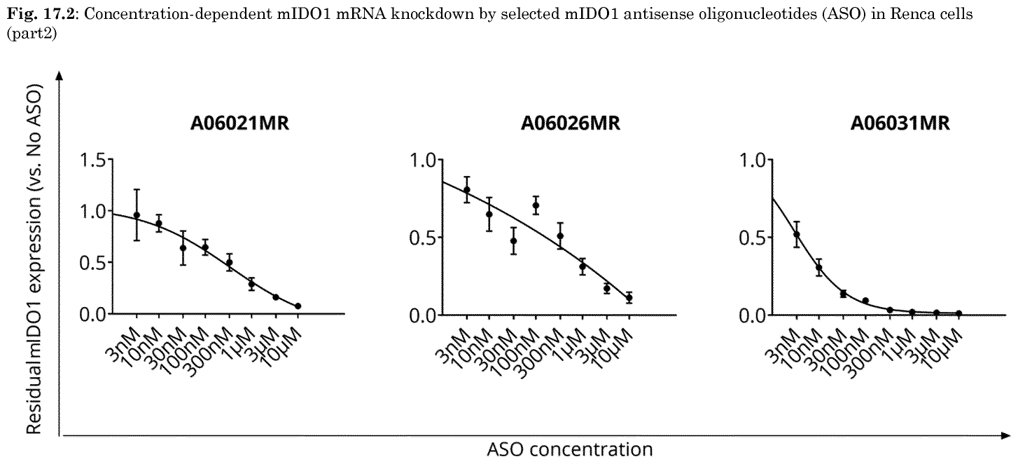

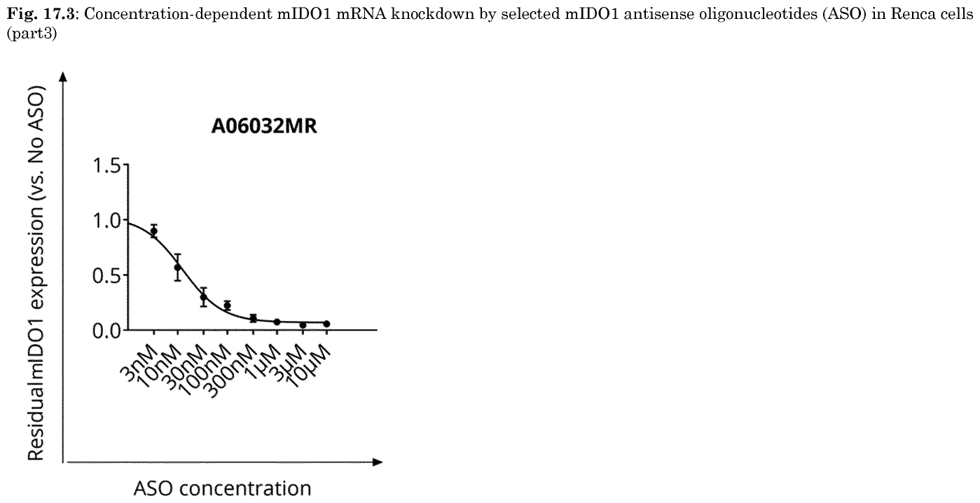

In order to determine the IC₅₀ of the mIDO1 antisense oligonucleotides A06013MR (SEQ ID No. 74), A06019MR (SEQ ID No. 80), A06020MR (SEQ ID No. 81), A06021MR (SEQ ID No. 82), A06026MR (SEQ ID No. 87), A06031MR (SEQ ID No. 60) and A06032MR (SEQ ID No. 61), Renca cells were treated with mIFNg to induce mIDO1 expression and titrated amounts of the respective antisense oligonucleotides (concentrations: 10 μM, 3 μM, 1 μM, 300 nM, 100 nM, 30 nM, 10 nM, 3 nM). mIDO1 mRNA expression was analyzed three days later. As shown in FIG. 17 and in the following Table 15, the antisense oligonucleotides A06031MR (SEQ ID No. 60) and A06032MR (SEQ ID No. 61) had the highest potency in Renca cells with regard to downregulation of mIDO1 mRNA compared to untreated cells with a maximal target inhibition of 98.9% and 97.3%, respectively. Table 19 shows IC₅₀ values and target inhibition of selected antisense oligonucleotides at titrated concentrations in Renca cells:

TABLE 19
Overview of IC₅₀ values of mIDO1 antisense oligonucleotides.

| | IC₅₀ | Inhibition (%) | | | | | | | |
|---|---|---|---|---|---|---|---|---|---|
| ASO | (nM) | 10 μM | 3 μM | 1 μM | 300 nM | 100 nM | 30 nM | 10 nM | 3 nM |
| A06013MR | 104 | 97.3 | 95.4 | 85.7 | 65.2 | 50.1 | 32.3 | 21.5 | 0 |
| A06019MR | 94 | 95.3 | 92.3 | 86.1 | 71.1 | 49.6 | 30.8 | 9.5 | 7 |
| A06020MR | n/a | 82.9 | 45.9 | 36.5 | 13 | 34.7 | 13.3 | 2.4 | 14.9 |
| A06021MR | 345 | 92.4 | 83.9 | 71.2 | 50 | 35.4 | 36.1 | 12.2 | 4.2 |
| A06026MR | n/a | 88.8 | 82.9 | 68.9 | 49.1 | 29.5 | 52.3 | 35.2 | 19.4 |
| A06031MR | 3 | 98.9 | 98.4 | 98 | 96.7 | 90.6 | 86.3 | 69.3 | 48.1 |
| A06032MR | 13 | 97.3 | 95.4 | 85.7 | 65.2 | 50.1 | 32.3 | 21.5 | 0 |

### Example 16: ASO-Mediated In Vivo mIDO1 Knockdown in a Syngeneic Mouse Tumor Model

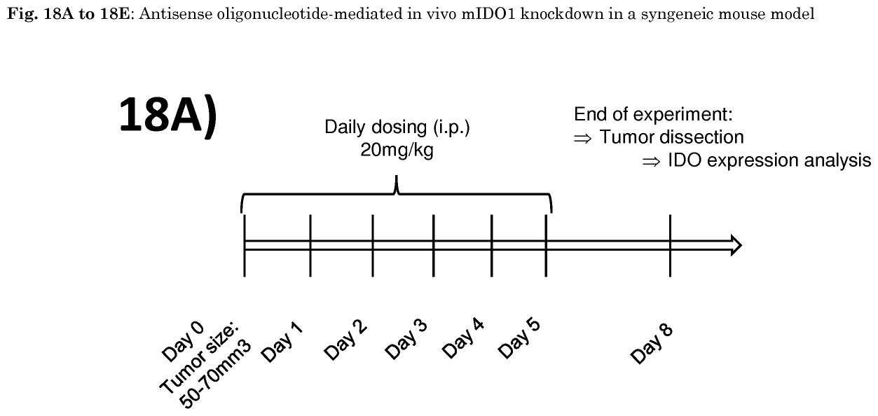

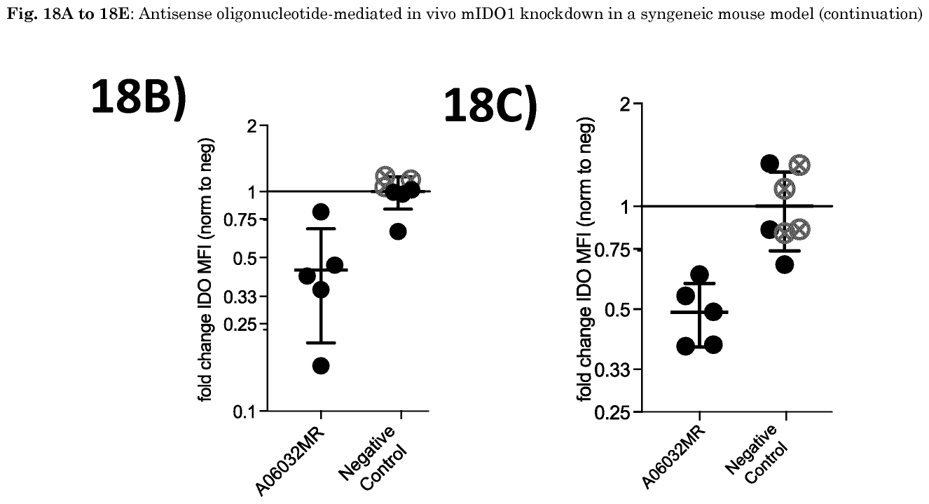

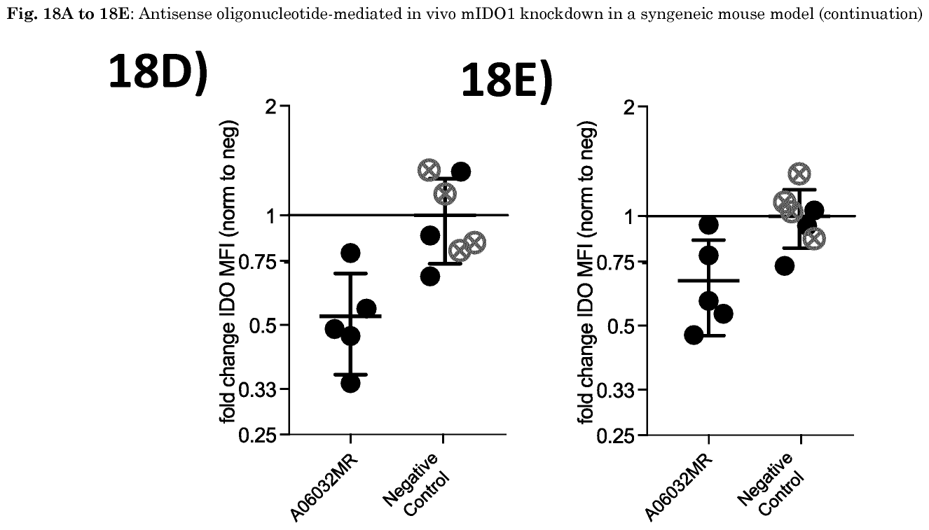

The in vivo knockdown capacity of mIDO1 antisense oligonucleotide A06032MR (SEQ ID No. 61) was analyzed in a subcutaneous syngeneic murine tumor model. Therefore, MC-38 cells were injected subcutaneously into C57BL/6 mice. After the tumors had reached a size of 50-70 mm³, mice were treated with the control antisense oligonucleotide neg1 or the mIDO1-specific antisense oligonucleotide A06032MR for 5 days by daily intraperitoneal injection of 20 mg/kg without the use of a delivery agent. Mice were sacrificed on day 8 and single cell suspensions of tumors were prepared after tumor resection (experimental setup: FIG. 18A).

The knockdown of mIDO1 on the protein level was investigated in different cells types by flow cytometry. Strikingly, a ˜50% knockdown of IDO1 was observed in tumor cells (FIG. 18B), monocytic myeloid-derived suppressor cells (M-MDSC) (FIG. 18C) and tumor-associated macrophages (FIG. 18D). Further, a knockdown of ˜30% was observed in granulocytic myeloid-derived suppressor cells (G-MDSC) (FIG. 18E).

1. An immunosuppression-reverting oligonucleotide comprising 12 to 18 nucleotides, wherein at least one of the nucleotides is modified, and the oligonucleotide hybridizes with a nucleic acid sequence of indoleamine-2,3-dioxygenase (IDO-1) of SEQ ID NO.1 (human) in a hybridizing active area wherein the oligonucleotide inhibits at least 50% of the IDO-1 expression.

2. The oligonucleotide of claim 1, wherein the hybridizing active area is selected from position 300 to 360, position 250 to 455, position 100 to 160, position 245 to 305, and/or position 650 to 710 of SEQ ID NO. 1.

3. The oligonucleotide of claim 1, wherein the modified nucleotide is selected from the group consisting of a bridged nucleic acid such as LNA, cET, ENA, 2′Fluoro modified nucleotide, 2′O-Methyl modified nucleotide and a combination thereof.

4. The oligonucleotide of claim 1 hybridizing with IDO-1 of SEQ ID NO.1 comprising a sequence selected from the group consisting of SEQ ID NO.3, SEQ ID NO.93, SEQ ID NO.94, SEQ ID NO.99, SEQ ID NO.105, SEQ ID NO.107, SEQ ID NO.101, SEQ ID NO.4, SEQ ID NO.95, SEQ ID NO.102, SEQ ID NO.96, SEQ ID NO.11, SEQ ID NO.97, SEQ ID NO.103, SEQ ID NO.104, SEQ ID NO.108, SEQ ID NO.109, SEQ ID NO.37, SEQ ID NO.100, SEQ ID NO.106 and a combination thereof.

5. The oligonucleotide of claim 1, wherein the oligonucleotide is selected from the group consisting of
+A*+G*+G*C*G*C*T*G*T*G*A*C*T*+T*+G*+T (A06030H),
+G*+C*G*C*T*G*T*G*A*C*T*+T*+G*+T (A06057H),
+T*+G*+T*C*C*C*G*T*T*C*T*+T*+G*+C (A06058H),
+A*+G*+G*C*G*C*T*G*T*G*A*C*T*+T*+G (A06062H),
+A*+G*+G*C*G*C*T*G*T*G*A*C*T*T*+G*+T (A06068H),
+G*+A*+T*T*G*T*C*C*A*G*G*A*G*T*+T*+T*+T (A06070H),
+G*+A*T*T*G*T*C*C*A*G*G*A*+G*+T*+T (A06059H),
+T*+G*+A*T*T*G*T*C*C*A*G*G*A*+G*+T*+T (A06065H),
+T*+G*+A*T*T*G*T*C*C*A*G*G*+A*+G*+T (A06060H),
+C*+T*+C*A*A*C*T*C*T*T*T*C*+T*+C*+G (A06008H),
+C*T*+C*A*A*C*T*C*T*T*T*C*+T*+C*+G (A06061H),
+T*+C*+T*C*A*A*C*T*C*T*T*T*C*+T*+C*+G (A06066H),
+T*+T*+C*T*C*A*A*C*T*C*T*T*T*+C*+T*+C (A06067H),
+C*+T*+C*A*A*C*T*C*T*T*T*C*T*C*+G*+A*+A (A06071H),
C*+T*+C*+A*A*C*T*C*T*T*T*C*T*C*+G*+A*+A (A06072H),
+A*+G*+T*G*T*C*C*C*G*T*T*C*T*+T*+G*+C (A06035H),
+G*+T*+G*T*C*C*C*G*T*T*C*T*+T*+G*+C (A06063H),
+A*+G*+T*G*T*C*C*C*G*T*T*C*T*T*+G*+C (A06069H),
and a combination thereof, wherein + indicates an LNA nucleotide and * indicates a phosphorothioate (PTO) linkage between the nucleotides.

6. The oligonucleotide of claim 1, wherein the oligonucleotide inhibits the expression of IDO-1 at a nanomolar concentration.

7. A pharmaceutical composition comprising an immunosuppression-reverting oligonucleotide of claim 1 and a pharmaceutically acceptable carrier, excipient, dilutant or a combination thereof.

8. The pharmaceutical composition of claim 7, further comprising a chemotherapeutic agent selected from the group consisting of platinum, gemcitabine, another oligonucleotide, an antibody, a small molecule, and a combination thereof.

9. The pharmaceutical composition of claim 7, wherein the other oligonucleotide, the antibody and/or the small molecule inhibits or stimulates an immune suppressive factor and/or an immune stimulatory factor.

10. The pharmaceutical composition of claim 9, wherein the immune suppressive factor is selected from the group consisting of IDO1, IDO2, CTLA-4, PD-1, PD-L1, LAG-3, VISTA, A2AR, CD39, CD73, STAT3, TDO2, TIM-3, TIGIT, TGF-beta, BTLA, MICA, NKG2A, KIR, CD160, Chop, Xbp1 and a combination thereof.

11. The pharmaceutical composition of claim 9, wherein the immune stimulatory factor is selected from the group consisting of 4-1BB, Ox40, KIR, GITR, CD27, 2B4 and a combination thereof.

12. A method of preventing and/or treating a disorder, where an IDO imbalance is involved, comprising administering to a subject in need thereof the immunosuppression-reverting oligonucleotide of claim 1.

13. The method according to claim 12, wherein the disorder is an autoimmune disorder, an immune disorder, a psychiatric disorder and/or cancer.

14. The method according to claim 13, wherein the cancer is breast cancer, lung cancer, malignant melanoma, lymphoma, skin cancer, bone cancer, prostate cancer, liver cancer, brain cancer, cancer of the larynx, gall bladder, pancreas, testicular, rectum, parathyroid, thyroid, adrenal, neural tissue, head and neck, colon, stomach, bronchi, kidneys, basal cell carcinoma, squamous cell carcinoma, metastatic skin carcinoma, osteo sarcoma, Ewing's sarcoma, reticulum cell sarcoma, liposarcoma, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilm's tumor, seminoma, ovarian tumor, leiomyomater tumor, cervical dysplasia, retinoblastoma, soft tissue sarcoma, malignant carcinoid, topical skin lesion, rhabdomyosarcoma, Kaposi's sarcoma, osteogenic sarcoma, malignant hypercalcemia, renal cell tumor, polycythermia vera, adenocarcinoma, anaplastic astrocytoma, glioblastoma multiforma, leukemia, or epidermoid carcinoma.

15. The method according to claim 12, wherein the oligonucleotide or the composition is suitable to be administered locally or systemically.


SEQUENCE LISTING (111 sequences)

SEQ ID NO: 1 - DNA, 1944 bp, Homo sapiens
aatttctcactgcccctgtgataaactgtggtcactggctgtggcagcaactattataagatgctctgaaaactcttcagacactgaggggcaccagaggagcagactacaagaatggcacacgctatggaaaactcctggacaatcagtaaagagtaccatattgatgaagaagtgggctttgctctgccaaatccacaggaaaatctacctgatttttataatgactggatgttcattgctaaacatctgcctgatctcatagagtctggccagcttcgagaaagagttgagaagttaaacatgctcagcattgatcatctcacagaccacaagtcacagcgccttgcacgtctagttctgggatgcatcaccatggcatatgtgtggggcaaaggtcatggagatgtccgtaaggtcttgccaagaaatattgctgttccttactgccaactctccaagaaactggaactgcctcctattttggtttatgcagactgtgtcttggcaaactggaagaaaaaggatcctaataagcccctgacttatgagaacatggacgttttgttctcatttcgtgatggagactgcagtaaaggattcttcctggtctctctattggtggaaatagcagctgcttctgcaatcaaagtaattcctactgtattcaaggcaatgcaaatgcaagaacgggacactttgctaaaggcgctgttggaaatagcttcttgcttggagaaagcccttcaagtgtttcaccaaatccacgatcatgtgaacccaaaagcatttttcagtgttcttcgcatatatttgtctggctggaaaggcaacccccagctatcagacggtctggtgtatgaagggttctgggaagacccaaaggagtttgcagggggcagtgcaggccaaagcagcgtctttcagtgctttgacgtcctgctgggcatccagcagactgctggtggaggacatgctgctcagttcctccaggacatgagaagatatatgccaccagctcacaggaacttcctgtgctcattagagtcaaatccctcagtccgtgagtttgtcctttcaaaaggtgatgctggcctgcgggaagcttatgacgcctgtgtgaaagctctggtctccctgaggagctaccatctgcaaatcgtgactaagtacatcctgattcctgcaagccagcagccaaaggagaataagacctctgaagacccttcaaaactggaagccaaaggaactggaggcactgatttaatgaatttcctgaagactgtaagaagtacaactgagaaatcccttttgaaggaaggttaatgtaacccaacaagagcacattttatcatagcagagacatctgtatgcattcctgtcattacccattgtaacagagccacaaactaatactatgcaatgttttaccaataatgcaatacaaaagacctcaaaatacctgtgcatttcttgtaggaaaacaacaaaaggtaattatgtgtaattatactagaagttttgtaatctgtatcttatcattggaataaaatgacattcaataaataaaaatgcataagatatattctgtcggctgggcgcggtggctcacgcctgtaatcccagcactttgggaggccgaggcgggcggatcacaaggtcaggagatcgagaccatcttggctaacacggtgaaaccccgtctctactaaaaatacaaaaaattagccgggcgcggtggcgggcacctgtagtcccagctactcgggaggctgaggcaggagaatggcgtgaacctgggaggcggagcttgcagtgagccaagattgtgccactgcaatccggcctgggctaaagagcgggactccgtctcaaaaaaaaaaaaaaaaagatatattctgtcataataaataaaaatgcataagatataaaaaaaaaaaaaaa

SEQ ID NO: 2 - DNA, 1506 bp, Mus musculus
ggagtagacagcaatggcactcagtaaaatatctcctacagaaggttctagaaggatccttgaagaccaccacatagatgaagatgtgggctttgctctaccacatccactggtggagctgcccgacgcatacagcccctgggtccttgtggctagaaatctgcctgtgctgattgagaacgggcagcttcgagaagaagttgaaaagctgcccacactgagcacggacggactgagaggacacaggttacagcgcctggcacacctggccctggggtacatcaccatggcgtatgtgtggaaccgaggggatgacgatgttcgaaaggtgctgccccgcaatattgctgttccctactgcgagctctcagagaagttgggcctgcctcctattctgtcttatgcagactgtgtcctggcaaactggaagaaaaaggaccccaatgggcccatgacatacgagaacatggacattctgttctcatttcctggtggggactgcgacaagggcttcttcctcgtctctctattggtggaaatcgcagcttctcctgcaatcaaagcaatccccactgtatccagtgcagtagagcgtcaagacctgaaagcattggaaaaggcactgcacgacatagctaccagtctggagaaagccaaggaaatttttaagaggatgcgtgactttgtggacccagacacgtttttccacgttctccgcatatatctgtctggctggaaatgcagctccaagctgccagaaggtctgctgtatgagggggtctgggacaccccaaaaatgttttcagggggcagtgcaggccagagcagcatcttccagagtcttgatgtccttctgggaataaaacacgaggctggcaaagaatctcctgcagaattcctccaggaaatgagagagtacatgcctccagcccaccggaacttccttttcttcttagagtcagctcccccagtccgtgagtttgtcatttcaagacacaatgaagacttgacgaaagcttataacgagtgtgtgaatggtctggtctctgtgagaaagttccacctcgcaatagtagatacttacattatgaaaccttcgaagaagaagcccactgatggcgacaagtcggaagagccctcaaatgtggaaagcagagggactgggggtacgaatcccatgactttcctaaggagtgtgaaagatacaaccgagaaagctcttctgagttggccttagtgtagcaagctccacttctatcagggcacagaaaacaccttcatcctgtcatagctcattaaatcagatccaccaagtaagactatagaataatttgcctgttgtatattatgtagatttcaaaaatcatctgtgcattccctgtaggaaaataatcaagctgaactatttaatgatattaaaatgatataagaaataatataaaatatatttatgattgaaatacaacaatgagacccagtaaataaaagttattgtgaagttgacta

SEQ ID NO: 3 - DNA, 16 bp, Artificial Sequence
  antisense nucleotide hybridizing with human IDO1
aggcgctgtgacttgt

SEQ ID NO: 4 - DNA, 15 bp, Artificial Sequence
  antisense nucleotide hybridizing with human IDO1
gattgtccaggagtt

SEQ ID NO: 5 - DNA, 14 bp, Artificial Sequence
  antisense nucleotide hybridizing with human IDO1
ggcgctgtgacttg

SEQ ID NO: 6 - DNA, 14 bp, Artificial Sequence
  antisense nucleotide hybridizing with human IDO1
aggcgctgtgactt

SEQ ID NO: 7 - DNA, 14 bp, Artificial Sequence
  antisense nucleotide hybridizing with human IDO1
atgcgaagaacact

SEQ ID NO: 8 - DNA, 14 bp, Artificial Sequence
  antisense nucleotide hybridizing with human IDO1
tatatgcgaagaac

SEQ ID NO: 9 - DNA, 14 bp, Artificial Sequence
  antisense nucleotide hybridizing with human IDO1
acttagtcacgatt

SEQ ID NO: 10 - DNA, 15 bp, Artificial Sequence
  antisense nucleotide hybridizing with human IDO1
gccattcttgtagtc

SEQ ID NO: 11 - DNA, 15 bp, Artificial Sequence
  antisense nucleotide hybridizing with human IDO1
ctcaactctttctcg

SEQ ID NO: 12 - DNA, 15 bp, Artificial Sequence
  antisense nucleotide hybridizing with human IDO1
ggcgctgtgacttgt

SEQ ID NO: 13 - DNA, 15 bp, Artificial Sequence
  antisense nucleotide hybridizing with human IDO1
ttggcaagaccttac

SEQ ID NO: 14 - DNA, 15 bp, Artificial Sequence
  antisense nucleotide hybridizing with human IDO1
gttggcagtaaggaa

SEQ ID NO: 15 - DNA, 15 bp, Artificial Sequence
  antisense nucleotide hybridizing with human IDO1
gacacagtctgcata

SEQ ID NO: 16 - DNA, 15 bp, Artificial Sequence
  antisense nucleotide hybridizing with human IDO1
gtcaggggcttatta

SEQ ID NO: 17 - DNA, 15 bp, Artificial Sequence
  antisense nucleotide hybridizing with human IDO1
gagaacaaaacgtcc

SEQ ID NO: 18 - DNA, 15 bp, Artificial Sequence
  antisense nucleotide hybridizing with human IDO1
agtgtcccgttcttg

SEQ ID NO: 19 - DNA, 15 bp, Artificial Sequence
  antisense nucleotide hybridizing with human IDO1
tatgcgaagaacact

SEQ ID NO: 20 - DNA, 15 bp, Artificial Sequence
  antisense nucleotide hybridizing with human IDO1
aggacgtcaaagcac

SEQ ID NO: 21 - DNA, 15 bp, Artificial Sequence
  antisense nucleotide hybridizing with human IDO1
gagctggtggcatat

SEQ ID NO: 22 - DNA, 15 bp, Artificial Sequence
  antisense nucleotide hybridizing with human IDO1
gagctggtggcatat

SEQ ID NO: 23 - DNA, 15 bp, Artificial Sequence
  antisense nucleotide hybridizing with human IDO1
gacaaactcacggac

SEQ ID NO: 24 - DNA, 15 bp, Artificial Sequence
  antisense nucleotide hybridizing with human IDO1
gacaaactcacggac

SEQ ID NO: 25 - DNA, 15 bp, Artificial Sequence
  antisense nucleotide hybridizing with human IDO1
gacaaactcacggac

SEQ ID NO: 26 - DNA, 15 bp, Artificial Sequence
  antisense nucleotide hybridizing with human IDO1
taagcttcccgcagg

SEQ ID NO: 27 - DNA, 15 bp, Artificial Sequence
  antisense nucleotide hybridizing with human IDO1
agatggtagctcctc

SEQ ID NO: 28 - DNA, 15 bp, Artificial Sequence
  antisense nucleotide hybridizing with human IDO1
gtacttagtcacgat

SEQ ID NO: 29 - DNA, 15 bp, Artificial Sequence
  antisense nucleotide hybridizing with human IDO1
tggcttgcaggaatc

SEQ ID NO: 30 - DNA, 15 bp, Artificial Sequence
  antisense nucleotide hybridizing with human IDO1
gtcttcagaggtctt

SEQ ID NO: 31 - DNA, 16 bp, Artificial Sequence
  antisense nucleotide hybridizing with human IDO1
cttgtagtctgctcct

SEQ ID NO: 32 - DNA, 16 bp, Artificial Sequence
  antisense nucleotide hybridizing with human IDO1
ggcgctgtgacttgtg

SEQ ID NO: 33 - DNA, 16 bp, Artificial Sequence
  antisense nucleotide hybridizing with human IDO1
gcaagaccttacggac

SEQ ID NO: 34 - DNA, 16 bp, Artificial Sequence
  antisense nucleotide hybridizing with human IDO1
gttggcagtaaggaac

SEQ ID NO: 35 - DNA, 16 bp, Artificial Sequence
  antisense nucleotide hybridizing with human IDO1
gagaacaaaacgtcca

SEQ ID NO: 36 - DNA, 16 bp, Artificial Sequence
  antisense nucleotide hybridizing with human IDO1
cagtctccatcacgaa

SEQ ID NO: 37 - DNA, 16 bp, Artificial Sequence
  antisense nucleotide hybridizing with human IDO1
agtgtcccgttcttgc

SEQ ID NO: 38 - DNA, 16 bp, Artificial Sequence
  antisense nucleotide hybridizing with human IDO1
aatatatgcgaagaac

SEQ ID NO: 39 - DNA, 16 bp, Artificial Sequence
  antisense nucleotide hybridizing with human IDO1
caggacgtcaaagcac

SEQ ID NO: 40 - DNA, 16 bp, Artificial Sequence
  antisense nucleotide hybridizing with human IDO1
tgagctggtggcatat

SEQ ID NO: 41 - DNA, 16 bp, Artificial Sequence
  antisense nucleotide hybridizing with human IDO1
gacaaactcacggact

SEQ ID NO: 42 - DNA, 16 bp, Artificial Sequence
  antisense nucleotide hybridizing with human IDO1
ttgcagatggtagctc

SEQ ID NO: 43 - DNA, 16 bp, Artificial Sequence
  antisense nucleotide hybridizing with human IDO1
gaggtcttttgtattg

SEQ ID NO: 44 - DNA, 17 bp, Artificial Sequence
  antisense nucleotide hybridizing with human IDO1
attcttgtagtctgctc

SEQ ID NO: 45 - DNA, 17 bp, Artificial Sequence
  antisense nucleotide hybridizing with human IDO1
ccagactctatgagatc

SEQ ID NO: 46 - DNA, 17 bp, Artificial Sequence
  antisense nucleotide hybridizing with human IDO1
gagatgatcaatgctga

SEQ ID NO: 47 - DNA, 17 bp, Artificial Sequence
  antisense nucleotide hybridizing with human IDO1
aggcgctgtgacttgtg

SEQ ID NO: 48 - DNA, 17 bp, Artificial Sequence
  antisense nucleotide hybridizing with human IDO1
ggtgatgcatcccagaa

SEQ ID NO: 49 - DNA, 17 bp, Artificial Sequence
  antisense nucleotide hybridizing with human IDO1
ggcaagaccttacggac

SEQ ID NO: 50 - DNA, 17 bp, Artificial Sequence
  antisense nucleotide hybridizing with human IDO1
gttggcagtaaggaaca

SEQ ID NO: 51 - DNA, 17 bp, Artificial Sequence
  antisense nucleotide hybridizing with human IDO1
acaaaacgtccatgttc

SEQ ID NO: 52 - DNA, 17 bp, Artificial Sequence
  antisense nucleotide hybridizing with human IDO1
agtgtcccgttcttgca

SEQ ID NO: 53 - DNA, 17 bp, Artificial Sequence
  antisense nucleotide hybridizing with human IDO1
gaactgagcagcatgtc

SEQ ID NO: 54 - DNA, 17 bp, Artificial Sequence
  antisense nucleotide hybridizing with human IDO1
gagctggtggcatatat

SEQ ID NO: 55 - DNA, 17 bp, Artificial Sequence
  antisense nucleotide hybridizing with human IDO1
gttcctgtgagctggtg

SEQ ID NO: 56 - DNA, 17 bp, Artificial Sequence
  antisense nucleotide hybridizing with human IDO1
aggacaaactcacggac

SEQ ID NO: 57 - DNA, 17 bp, Artificial Sequence
  antisense nucleotide hybridizing with human IDO1
ccgcaggccagcatcac

SEQ ID NO: 58 - DNA, 17 bp, Artificial Sequence
  antisense nucleotide hybridizing with human IDO1
ttgcagatggtagctcc

SEQ ID NO: 59 - DNA, 18 bp, Artificial Sequence
  negative control
cgtttaggctatgtactt

SEQ ID NO: 60 - DNA, 17 bp, Artificial Sequence
  antisense oligonucleotide hybridizing with rat or murine IDO1
tgtatctttcacactcc

SEQ ID NO: 61 - DNA, 17 bp, Artificial Sequence
  antisense oligonucleotide hybridizing with rat or murine IDO1
gttgtatctttcacact

SEQ ID NO: 62 - DNA, 15 bp, Artificial Sequence
  antisense oligonucleotide hybridizing with rat or murine IDO1
ggcgctgtaacctgt

SEQ ID NO: 63 - DNA, 15 bp, Artificial Sequence
  antisense oligonucleotide hybridizing with rat or murine IDO1
tcggttccacacata

SEQ ID NO: 64 - DNA, 15 bp, Artificial Sequence
  antisense oligonucleotide hybridizing with rat or murine IDO1
tcccctcggttccac

SEQ ID NO: 65 - DNA, 15 bp, Artificial Sequence
  antisense oligonucleotide hybridizing with rat or murine IDO1
gtccatgttctcgta

SEQ ID NO: 66 - DNA, 15 bp, Artificial Sequence
  antisense oligonucleotide hybridizing with rat or murine IDO1
tcgcagtccccacca

SEQ ID NO: 67 - DNA, 15 bp, Artificial Sequence
  antisense oligonucleotide hybridizing with rat or murine IDO1
gagaagctgcgattt

SEQ ID NO: 68 - DNA, 15 bp, Artificial Sequence
  antisense oligonucleotide hybridizing with rat or murine IDO1
tcacgcatcctctta

SEQ ID NO: 69 - DNA, 15 bp, Artificial Sequence
  antisense oligonucleotide hybridizing with rat or murine IDO1
aagtcacgcatcctc

SEQ ID NO: 70 - DNA, 16 bp, Artificial Sequence
  antisense oligonucleotide hybridizing with rat or murine IDO1
aggcgctgtaacctgt

SEQ ID NO: 71 - DNA, 16 bp, Artificial Sequence
  antisense oligonucleotide hybridizing with rat or murine IDO1
tcggttccacacatac

SEQ ID NO: 72 - DNA, 16 bp, Artificial Sequence
  antisense oligonucleotide hybridizing with rat or murine IDO1
catcccctcggttcca

SEQ ID NO: 73 - DNA, 16 bp, Artificial Sequence
  antisense oligonucleotide hybridizing with rat or murine IDO1
ggcagcacctttcgaa

SEQ ID NO: 74 - DNA, 16 bp, Artificial Sequence
  antisense oligonucleotide hybridizing with rat or murine IDO1
gagagctcgcagtagg

SEQ ID NO: 75 - DNA, 16 bp, Artificial Sequence
  antisense oligonucleotide hybridizing with rat or murine IDO1
tgtccatgttctcgta

SEQ ID NO: 76 - DNA, 16 bp, Artificial Sequence
  antisense oligonucleotide hybridizing with rat or murine IDO1
tcgcagtccccaccag

SEQ ID NO: 77 - DNA, 16 bp, Artificial Sequence
  antisense oligonucleotide hybridizing with rat or murine IDO1
aagctgcgatttccac

SEQ ID NO: 78 - DNA, 16 bp, Artificial Sequence
  antisense oligonucleotide hybridizing with rat or murine IDO1
agtcacgcatcctctt

SEQ ID NO: 79 - DNA, 16 bp, Artificial Sequence
  antisense oligonucleotide hybridizing with rat or murine IDO1
tgacaaactcacggac

SEQ ID NO: 80 - DNA, 16 bp, Artificial Sequence
  antisense oligonucleotide hybridizing with rat or murine IDO1
gttgtatctttcacac

SEQ ID NO: 81 - DNA, 17 bp, Artificial Sequence
  antisense oligonucleotide hybridizing with rat or murine IDO1
agtggatgtggtagagc

SEQ ID NO: 82 - DNA, 17 bp, Artificial Sequence
  antisense oligonucleotide hybridizing with rat or murine IDO1
aggcgctgtaacctgtg

SEQ ID NO: 83 - DNA, 17 bp, Artificial Sequence
  antisense oligonucleotide hybridizing with rat or murine IDO1
tcggttccacacatacg

SEQ ID NO: 84 - DNA, 17 bp, Artificial Sequence
  antisense oligonucleotide hybridizing with rat or murine IDO1
cctcggttccacacata

SEQ ID NO: 85 - DNA, 17 bp, Artificial Sequence
  antisense oligonucleotide hybridizing with rat or murine IDO1
atgtccatgttctcgta

SEQ ID NO: 86 - DNA, 17 bp, Artificial Sequence
  antisense oligonucleotide hybridizing with rat or murine IDO1
tcgcagtccccaccagg

SEQ ID NO: 87 - DNA, 17 bp, Artificial Sequence
  antisense oligonucleotide hybridizing with rat or murine IDO1
attgctttgattgcagg

SEQ ID NO: 88 - DNA, 17 bp, Artificial Sequence
  antisense oligonucleotide hybridizing with rat or murine IDO1
gtcacgcatcctcttaa

SEQ ID NO: 89 - DNA, 17 bp, Artificial Sequence
  antisense oligonucleotide hybridizing with rat or murine IDO1
agtcacgcatcctctta

SEQ ID NO: 90 - DNA, 17 bp, Artificial Sequence
  antisense oligonucleotide hybridizing with rat or murine IDO1
gaaggacatcaagactc

SEQ ID NO: 91 - DNA, 17 bp, Artificial Sequence
  antisense oligonucleotide hybridizing with rat or murine IDO1
gctggaggcatgtactc

SEQ ID NO: 92 - DNA, 18 bp, Artificial Sequence
  negative control
cgtttaggctatgtactt

SEQ ID NO: 93 - DNA, 14 bp, Artificial Sequence
  antisense nucleotide hybridizing with human IDO1
gcgctgtgacttgt

SEQ ID NO: 94 - DNA, 14 bp, Artificial Sequence
  antisense nucleotide hybridizing with human IDO1
gcgctgtgacttgt

SEQ ID NO: 95 - DNA, 15 bp, Artificial Sequence
  antisense nucleotide hybridizing with human IDO1
gattgtccaggagtt

SEQ ID NO: 96 - DNA, 15 bp, Artificial Sequence
  antisense nucleotide hybridizing with human IDO1
tgattgtccaggagt

SEQ ID NO: 97 - DNA, 15 bp, Artificial Sequence
  antisense nucleotide hybridizing with human IDO1
ctcaactctttctcg

SEQ ID NO: 98 - DNA, 20 bp, Artificial Sequence
  fake sequence to secure correct SEQ ID NOs
aaaaaaaaaaaaaaaaaaaa

SEQ ID NO: 99 - DNA, 15 bp, Artificial Sequence
  antisense nucleotide hybridizing with human IDO1
aggcgctgtgacttg

SEQ ID NO: 100 - DNA, 15 bp, Artificial Sequence
  antisense nucleotide hybridizing with human IDO1
gtgtcccgttcttgc

SEQ ID NO: 101 - DNA, 16 bp, Artificial Sequence
  antisense nucleotide hybridizing with human IDO1
gattgtccaggagttt

SEQ ID NO: 102 - DNA, 16 bp, Artificial Sequence
  antisense nucleotide hybridizing with human IDO1
tgattgtccaggagtt

SEQ ID NO: 103 - DNA, 16 bp, Artificial Sequence
  antisense nucleotide hybridizing with human IDO1
tctcaactctttctcg

SEQ ID NO: 104 - DNA, 16 bp, Artificial Sequence
  antisense nucleotide hybridizing with human IDO1
ttctcaactctttctc

SEQ ID NO: 105 - DNA, 16 bp, Artificial Sequence
  antisense nucleotide hybridizing with human IDO1
aggcgctgtgacttgt

SEQ ID NO: 106 - DNA, 16 bp, Artificial Sequence
  antisense nucleotide hybridizing with human IDO1
agtgtcccgttcttgc

SEQ ID NO: 107 - DNA, 17 bp, Artificial Sequence
  antisense nucleotide hybridizing with human IDO1
gattgtccaggagtttt

SEQ ID NO: 108 - DNA, 17 bp, Artificial Sequence
  antisense nucleotide hybridizing with human IDO1
ctcaactctttctcgaa

SEQ ID NO: 109 - DNA, 17 bp, Artificial Sequence
  antisense nucleotide hybridizing with human IDO1
ctcaactctttctcgaa

SEQ ID NO: 110 - DNA, 17 bp, Artificial Sequence
  negative control AON
tctatcgtgatgtttct

SEQ ID NO: 111 - DNA, 18 bp, Artificial Sequence
  AON from WO2014154843
cgtttaggctatgtactt